SEQUENCE LISTING (123 sequences)

SEQ ID NO: 1 - DNA, 619 bp, homo sapiens
tgcagcttccttctcaccttgaagaataatcctagaaaactcacaaaatgtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataagccaagggacgttgacctggactgaagttcgcattgaactctacaacattctgtgggatatattgttcaaaaagatattgttgttttccctgatttagcaagcaagtaattttctcccaagctgattttattcaatatggttacgttggttaaataactttttttagatttag

SEQ ID NO: 2 - protein, 132 aa, homo sapiens
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 3 - DNA, 614 bp, mus musculus
cctttctcacctggaagacagctcctcctcgaaggtttacaaaatgtgtgatgcctttgtgggaacctggaagcttgtctccagtgaaaacttcgatgattacatgaaagaagtgggagtgggctttgccacaaggaaagtggcaggcatggccaagcccaacatgatcatcagcgtaaatggggatttggtcaccatccggtcagagagtacttttaaaaacaccgagatttccttcaaactgggcgtggaattcgatgaaatcaccgcagacgacaggaaggtgaagagcatcataaccctagatggcggggccctggtgcaggtgcagaagtgggatggaaagtcgaccacaataaagagaaaacgagatggtgacaagctggtggtggaatgtgttatgaaaggcgtgacttccacaagagtttatgaaagggcatgagccaaaggaagaggcctggatggaaatttgcatcaaacactacaatagtcagtcggatttattgttttttttaaagatatgattttccactaataagcaagcaattaattttttctgaagatgcattttattggatatggttatgttgattaaataaaacctttttagactt

SEQ ID NO: 4 - protein, 132 aa, mus musculus
MCDAFVGTWKLVSSENFDDYMKEVGVGFATRKVAGMAKPNMIISVNGDLVTIRSESTFKNTEISFKLGVEFDEITADDRKVKSIITLDGGALVQVQKWDGKSTTIKRKRDGDKLVVECVMKGVTSTRVYERA

SEQ ID NO: 5 - DNA, 16 bp, artificial
  Sequence motif
acaaaatgtgtgatgc

SEQ ID NO: 6 - DNA, 20 bp, artificial
  Sequence motif
cttgtctccagtgaaaactt

SEQ ID NO: 7 - DNA, 17 bp, artificial
  Sequence motif
ggagtgggctttgccac

SEQ ID NO: 8 - DNA, 17 bp, artificial
  Sequence motif
ataaagagaaaacgaga

SEQ ID NO: 9 - DNA, 14 bp, artificial
  Sequence motif
aacatgatcatcag

SEQ ID NO: 10 - DNA, 13 bp, artificial
  Sequence motif
gagatttccttca

SEQ ID NO: 11 - DNA, 14 bp, artificial
  Sequence motif
ctggtggtggaatg

SEQ ID NO: 12 - DNA, 14 bp, artificial
  Sequence motif
gcatcacacatttt

SEQ ID NO: 13 - DNA, 15 bp, artificial
  Sequence motif
ggcaaagcccactcc

SEQ ID NO: 14 - DNA, 15 bp, artificial
  Sequence motif
gtggcaaagcccact

SEQ ID NO: 15 - DNA, 15 bp, artificial
  Sequence motif
tcgttttctctttat

SEQ ID NO: 16 - DNA, 15 bp, artificial
  Sequence motif
ctcgttttctcttta

SEQ ID NO: 17 - DNA, 15 bp, artificial
  Sequence motif
gcatcacacattttg

SEQ ID NO: 18 - DNA, 15 bp, artificial
  Sequence motif
ttcactggagacaag

SEQ ID NO: 19 - DNA, 15 bp, artificial
  Sequence motif
tttcactggagacaa

SEQ ID NO: 20 - DNA, 16 bp, artificial
  Sequence motif
tggcaaagcccactcc

SEQ ID NO: 21 - DNA, 16 bp, artificial
  Sequence motif
gtggcaaagcccactc

SEQ ID NO: 22 - DNA, 16 bp, artificial
  Sequence motif
ctcgttttctctttat

SEQ ID NO: 23 - DNA, 16 bp, artificial
  Sequence motif
tctcgttttctcttta

SEQ ID NO: 24 - DNA, 16 bp, artificial
  Sequence motif
gcatcacacattttgt

SEQ ID NO: 25 - DNA, 16 bp, artificial
  Sequence motif
tttcactggagacaag

SEQ ID NO: 26 - DNA, 16 bp, artificial
  Sequence motif
ttttcactggagacaa

SEQ ID NO: 27 - DNA, 16 bp, artificial
  Sequence motif
gttttcactggagaca

SEQ ID NO: 28 - DNA, 14 bp, artificial
  Sequence motif
ctgatgatcatgtt

SEQ ID NO: 29 - DNA, 14 bp, artificial
  Sequence motif
cgttttctctttat

SEQ ID NO: 30 - DNA, 14 bp, artificial
  Sequence motif
ctcgttttctcttt

SEQ ID NO: 31 - DNA, 15 bp, artificial
  Sequence motif
tctcgttttctcttt

SEQ ID NO: 32 - DNA, 13 bp, artificial
  Sequence motif
tgaaggaaatctc

SEQ ID NO: 33 - DNA, 14 bp, artificial
  Sequence motif
tctcgttttctctt

SEQ ID NO: 34 - DNA, 14 bp, artificial
  Sequence motif
tcgttttctcttta

SEQ ID NO: 35 - DNA, 14 bp, artificial
  Sequence motif
catcacacattttg

SEQ ID NO: 36 - DNA, 14 bp, artificial
  Sequence motif
tttcactggagaca

SEQ ID NO: 37 - DNA, 14 bp, artificial
  Sequence motif
aagttttcactgga

SEQ ID NO: 38 - DNA, 15 bp, artificial
  Sequence motif
catcacacattttgt

SEQ ID NO: 39 - DNA, 15 bp, artificial
  Sequence motif
ttttcactggagaca

SEQ ID NO: 40 - DNA, 15 bp, artificial
  Sequence motif
gttttcactggagac

SEQ ID NO: 41 - DNA, 15 bp, artificial
  Sequence motif
agttttcactggaga

SEQ ID NO: 42 - DNA, 15 bp, artificial
  Sequence motif
aagttttcactggag

SEQ ID NO: 43 - DNA, 16 bp, artificial
  Sequence motif
agttttcactggagac

SEQ ID NO: 44 - DNA, 16 bp, artificial
  Sequence motif
aagttttcactggaga

SEQ ID NO: 45 - DNA, 14 bp, artificial
  Sequence motif
tggcaaagcccact

SEQ ID NO: 46 - DNA, 14 bp, artificial
  Sequence motif
atcacacattttgt

SEQ ID NO: 47 - DNA, 14 bp, artificial
  Sequence motif
agttttcactggag

SEQ ID NO: 48 - DNA, 15 bp, artificial
  Sequence motif
tggcaaagcccactc

SEQ ID NO: 49 - DNA, 13 bp, artificial
  Sequence motif
gcatcacacattt

SEQ ID NO: 50 - DNA, 14 bp, artificial
  Sequence motif
ggcaaagcccactc

SEQ ID NO: 51 - DNA, 14 bp, artificial
  Sequence motif
tcactggagacaag

SEQ ID NO: 52 - DNA, 14 bp, artificial
  Sequence motif
ttcactggagacaa

SEQ ID NO: 53 - DNA, 13 bp, artificial
  Sequence motif
cgttttctcttta

SEQ ID NO: 54 - DNA, 13 bp, artificial
  Sequence motif
attccaccaccag

SEQ ID NO: 55 - DNA, 14 bp, artificial
  Sequence motif
cattccaccaccag

SEQ ID NO: 56 - DNA, 13 bp, artificial
  Sequence motif
ctgatgatcatgt

SEQ ID NO: 57 - DNA, 13 bp, artificial
  Sequence motif
ctcgttttctctt

SEQ ID NO: 58 - DNA, 13 bp, artificial
  Sequence motif
tctcgttttctct

SEQ ID NO: 59 - DNA, 14 bp, artificial
  Sequence motif
ttttcactggagac

SEQ ID NO: 60 - DNA, 13 bp, artificial
  Sequence motif
atcacacattttg

SEQ ID NO: 61 - DNA, 14 bp, artificial
  Sequence motif
gttttcactggaga

SEQ ID NO: 62 - DNA, 13 bp, artificial
  Sequence motif
tcacacattttgt

SEQ ID NO: 63 - DNA, 13 bp, artificial
  Sequence motif
agttttcactgga

SEQ ID NO: 64 - DNA, 13 bp, artificial
  Sequence motif
tcgttttctcttt

SEQ ID NO: 65 - DNA, 13 bp, artificial
  Sequence motif
gttttcactggag

SEQ ID NO: 66 - DNA, 14 bp, artificial
  Sequence motif
gtggcaaagcccac

SEQ ID NO: 67 - DNA, 13 bp, artificial
  Sequence motif
tcactggagacaa

SEQ ID NO: 68 - DNA, 13 bp, artificial
  Sequence motif
tgatgatcatgtt

SEQ ID NO: 69 - DNA, 13 bp, artificial
  Sequence motif
aagttttcactgg

SEQ ID NO: 70 - DNA, 13 bp, artificial
  Sequence motif
ggcaaagcccact

SEQ ID NO: 71 - DNA, 12 bp, artificial
  Sequence motif
gcatcacacatt

SEQ ID NO: 72 - DNA, 12 bp, artificial
  Sequence motif
gaaggaaatctc

SEQ ID NO: 73 - DNA, 13 bp, artificial
  Sequence motif
tggcaaagcccac

SEQ ID NO: 74 - DNA, 13 bp, artificial
  Sequence motif
gttttctctttat

SEQ ID NO: 75 - DNA, 13 bp, artificial
  Sequence motif
catcacacatttt

SEQ ID NO: 76 - DNA, 12 bp, artificial
  Sequence motif
tctcgttttctc

SEQ ID NO: 77 - DNA, 13 bp, artificial
  Sequence motif
cattccaccacca

SEQ ID NO: 78 - DNA, 13 bp, artificial
  Sequence motif
tttcactggagac

SEQ ID NO: 79 - DNA, 13 bp, artificial
  Sequence motif
cactggagacaag

SEQ ID NO: 80 - DNA, 12 bp, artificial
  Sequence motif
ctcgttttctct

SEQ ID NO: 81 - DNA, 13 bp, artificial
  Sequence motif
ttcactggagaca

SEQ ID NO: 82 - DNA, 13 bp, artificial
  Sequence motif
gtggcaaagccca

SEQ ID NO: 83 - DNA, 12 bp, artificial
  Sequence motif
attccaccacca

SEQ ID NO: 84 - DNA, 12 bp, artificial
  Sequence motif
gatgatcatgtt

SEQ ID NO: 85 - DNA, 13 bp, artificial
  Sequence motif
ttttcactggaga

SEQ ID NO: 86 - DNA, 12 bp, artificial
  Sequence motif
ctgatgatcatg

SEQ ID NO: 87 - DNA, 12 bp, artificial
  Sequence motif
cgttttctcttt

SEQ ID NO: 88 - DNA, 12 bp, artificial
  Sequence motif
tcgttttctctt

SEQ ID NO: 89 - DNA, 12 bp, artificial
  Sequence motif
tgatgatcatgt

SEQ ID NO: 90 - DNA, 12 bp, artificial
  Sequence motif
tgaaggaaatct

SEQ ID NO: 91 - DNA, 12 bp, artificial
  Sequence motif
actggagacaag

SEQ ID NO: 92 - DNA, 12 bp, artificial
  Sequence motif
catcacacattt

SEQ ID NO: 93 - DNA, 12 bp, artificial
  Sequence motif
cactggagacaa

SEQ ID NO: 94 - DNA, 12 bp, artificial
  Sequence motif
aagttttcactg

SEQ ID NO: 95 - DNA, 12 bp, artificial
  Sequence motif
tcacacattttg

SEQ ID NO: 96 - DNA, 12 bp, artificial
  Sequence motif
cacacattttgt

SEQ ID NO: 97 - DNA, 12 bp, artificial
  Sequence motif
ggcaaagcccac

SEQ ID NO: 98 - DNA, 12 bp, artificial
  Sequence motif
gttttcactgga

SEQ ID NO: 99 - DNA, 12 bp, artificial
  Sequence motif
gtggcaaagccc

SEQ ID NO: 100 - DNA, 12 bp, artificial
  Sequence motif
tggcaaagccca

SEQ ID NO: 101 - DNA, 12 bp, artificial
  Sequence motif
agttttcactgg

SEQ ID NO: 102 - DNA, 12 bp, artificial
  Sequence motif
ttttcactggag

SEQ ID NO: 103 - DNA, 12 bp, artificial
  Sequence motif
cattccaccacc

SEQ ID NO: 104 - DNA, 12 bp, artificial
  Sequence motif
ttcactggagac

SEQ ID NO: 105 - DNA, 12 bp, artificial
  Sequence motif
atcacacatttt

SEQ ID NO: 106 - DNA, 12 bp, artificial
  Sequence motif
gttttctcttta

SEQ ID NO: 107 - DNA, 12 bp, artificial
  Sequence motif
tcactggagaca

SEQ ID NO: 108 - DNA, 14 bp, artificial
  Sequence motif
gcaaagcccactcc

SEQ ID NO: 109 - DNA, 12 bp, artificial
  Sequence motif
tttcactggaga

SEQ ID NO: 110 - DNA, 13 bp, artificial
  Sequence motif
caaagcccactcc

SEQ ID NO: 111 - DNA, 12 bp, artificial
  Sequence motif
ttccaccaccag

SEQ ID NO: 112 - DNA, 13 bp, artificial
  Sequence motif
gcaaagcccactc

SEQ ID NO: 113 - DNA, 12 bp, artificial
  Sequence motif
aaagcccactcc

SEQ ID NO: 114 - DNA, 12 bp, artificial
  Sequence motif
gcaaagcccact

SEQ ID NO: 115 - DNA, 12 bp, artificial
  Sequence motif
ttttctctttat

SEQ ID NO: 116 - DNA, 12 bp, artificial
  Sequence motif
caaagcccactc

SEQ ID NO: 117 - DNA, 15 bp, artificial
  LNA oligomer
tcgttttctctttat

SEQ ID NO: 118 - DNA, 16 bp, artificial
  Sequence motif
gcatcacacattttgt

SEQ ID NO: 119 - DNA, 15 bp, artificial
  Sequence motif
ttcactggagacaag

SEQ ID NO: 120 - DNA, 15 bp, artificial
  Sequence motif
tttcactggagacaa

SEQ ID NO: 121 - DNA, 16 bp, artificial
  Sequence motif
tctcgttttctcttta

SEQ ID NO: 122 - DNA, 14 bp, artificial
  Sequence motif
gcatcacacatttt

SEQ ID NO: 123 - DNA, 16 bp, artificial
  Sequence motif
gttttcactggagaca

(19) United States
(12) Patent Application Publication
(10) Pub. No.: US 2011/0054011 A1
(43) Pub. Date: Mar. 3, 2011

(54) RNA Antagonist Compounds for the Modulation of FABP4/AP2

(76) Inventors: Keith McCullagh, London (GB); Ellen Marie Straarup, Birkerod (DK); Niels Fisker Nielsen, Lyngby (DK)

(21) Appl. No.: 12/675,676

(22) PCT Filed: Aug. 29, 2008
(86) PCT No.: PCT/EP2008/061432

§ 371 Date: Oct. 14, 2010

Related U.S. Application Data
(60) Provisional application No. 60/969,016, filed on Aug. 30, 2007.

(51) Int. Cl. A61K 31/7088; C07H 21/00; C07H 21/04; C12N 5/071; A61P 3/04; A61P 5/50; A61P 3/10; A61P 3/00; A61P 29/00

(52) U.S. Cl. 514/44 R; 536/23.5; 435/375

(57) ABSTRACT

Oligonucleotides directed against the FABP4 gene are developed for modulating the expression of FABP4 protein. The compositions comprise oligonucleotides, particularly antisense oligonucleotides, targeted to nucleic acids encoding FABP4. Methods of using these compounds for modulation of FABP4 expression and for the treatment of diseases associated with over expression of FABP4 are provided. Examples of such diseases are the metabolic syndrome, diabetes, atherosclerosis, and inflammatory states such as arthritis. The oligomer may be composed of deoxyribonucleosides or a nucleic acid analogue such as for example locked nucleic acid (LNA) or a combination thereof.

### FIELD OF THE INVENTION

The present invention provides compounds, compositions and methods for modulating the expression of FABP4. In particular, this invention relates to oligomeric compounds (oligomers), which target the FABP4 mRNA in a cell, leading to reduced expression of FABP4. Reduction of FABP4 expression is beneficial for a range of metabolic and inflammatory disorders.

### BACKGROUND

The multi-gene family of intracellular lipid binding proteins includes more than thirty members differing in substrate affinity and tissue expression. The intracellular lipid binding proteins share a structural feature—a large hydrophobic internal cavity, providing a high affinity site for binding of fatty acids and other lipophilic biomolecules. A subclass of intracellular lipid binding proteins is named Fatty Acid Binding Proteins (FABPs). FABPs are involved in intracellular lipid transport, transfer of lipids across cell membranes, interaction with lipid metabolism enzymes, and possibly also protection of cell membranes against detergent effects of intracellular fatty acids (Hertzel and Bernlohr 2000). FABP sequestering of cytosolic unesterified fatty acids will protect cells against lipotoxicity, but the same mechanism may also hinder intracellular signaling. Long-chain unesterified fatty acids are ligands for nuclear receptors (Peroxisome Proliferator Related proteins; PPARs) and high expression of FABPs results in a blunted PPAR response, hindering the fatty acid nuclear receptor binding activity (Helledie et al. 2000).

Fatty acid binding protein 4 (FABP4, alternative names ALBP, Ap2, and Lbpl) is a 15 kDa protein with high level of expression in adipocytes and macrophages. The protein is considered to be involved in development of atherosclerosis and diabetes. This is supported by human epidemiological data, as well as in vitro experiments and data from FABP4 null mice. In humans, a FABP4 gene polymorphism has been identified that result in a lower FABP4 protein expression, which in turn is correlated with a significantly lower risk score for cardiovascular incidents and development of type 2 diabetes than in the general population. A low level of FABP4 mRNA expression has been detected in needle biopsies of adipose tissue from individuals with this FABP4 gene polymorphism, and low adipose tissue FABP4 protein content was correlated with increased insulin sensitivity (Tuncman et al. 2006). Mice lacking FABP4 (FABP4 null mice) lack gross morphological changes in response to the diet compared to wild-type animals. When kept on a high-fat diet, FABP4 null animals have lower plasma triglyceride, cholesterol, insulin, and glucose levels than wild-type animals, indicating that absence of FABP4 results a lower risk for development of diet-induced insulin resistance (Hotamisligil et al. 1996). When FABP4 null mice are crossed with a widely used model for atherosclerosis development, the apoE null mouse, results are dramatic. Loss of FABP4 appears to protect the animals from atherosclerotic development observed as a strong decrease in atherosclerotic plaque development (Makowski et al. 2001). The effect appears to be specific for macrophage, rather than adipocyte, expression of FABP4. Bone marrow transfer experiments have demonstrated a strong reduction in atherosclerotic plaque formation in animals lacking FABP4 in macrophages (Makowski, Boord, Maeda, Babaev, Uysal, Morgan, Parker, Suttles, Fazio, Hotamisligil, and Linton 2001).

Appearance of lipid-filled macrophages, i.e. macrophage foam cells, is a trademark of atherosclerosis development. The human monocyte/macrophage cell line THP-1 is a commonly used model for macrophage foam cell formation and inflammatory response. In vitro experiments with this cell type demonstrate that FABP4 promotes macrophage foam cell formation and macrophage inflammatory response; over-expression of FABP4 in THP-1 cells results in intracellular neutral lipid accumulation (cholesterol and triglycerides) concomitant with an up-regulation of proteins involved in intracellular lipid uptake and storage (SR-A1 and ACAT1), and down regulation of a protein involved in lipid efflux (ABCA1) (Fu et al. 2006). It has also been demonstrated that Toll like receptor agonists such as bacterial endotoxin strongly up-regulate macrophage FABP4 expression with concomitant increases in intracellular lipid droplet formation, further enhanced by co-incubation with oxidized LDL (Kazemi et al. 2005).

### SUMMARY OF THE INVENTION

The invention provides an oligomer of between 10-50 nucleobases in length which comprises a contiguous nucleobase sequence of a total of between 10-50 nucleobases, wherein said contiguous nucleobase sequence is at least 80% homologous to a corresponding region of a nucleic acid which encodes a mammalian FABP4.

In some embodiments, the oligomer is for use as a medicament or for use in a pharmaceutical composition.

The invention further provides a conjugate comprising the oligomer according to the invention, such as a conjugate which, in addition to the nucloebase sequence of the oligomer comprises at least one non-nucleotide or non-polynucleotide moiety covalently attached to the oligomer of the invention.

The invention provides for pharmaceutical composition comprising the oligomer or as defined conjugate of the invention, and a pharmaceutically acceptable diluent, carrier, salt or adjuvant.

The invention further provides for an oligomer according to the invention, for use in medicine.

The invention further provides for the use of the oligomer of the invention for the manufacture of a medicament for the treatment of one or more of the diseases referred to herein, such as a disease selected from the group consisting of: metabolic syndrome, diabetes, atherosclerosis, an inflammatory disease, arthritis, asthma and alzheimer's disease.

The invention further provides for an oligomer according to the invention, for use for the treatment of one or more of the diseases referred to herein, such as a disease selected from the group consisting of: metabolic syndrome, diabetes, atherosclerosis, an inflammatory disease, arthritis, asthma and alzheimer's disease.

The invention provides for a method for treating an inflammatory disorder such as arthritis, asthma or alzheimer's disease, said method comprising administering an oligomer, a conjugate, or a pharmaceutical composition according to the invention to a patient in need thereof.

The invention provides for a method of inhibiting or reducing the expression of FABP4 in a cell or a tissue, the method comprising the step of contacting said cell or tissue with an oligomer, a conjugate, or a pharmaceutical composition according to the invention so that expression of FABP4 is inhibited or reduced.

The invention provides for a method of (i) reducing the level of blood serum cholesterol or ii) reducing the level of blood serum LDL-cholesterol, or iii) for improving the HDL/LDL ratio, in a patient, the method comprising the step of administering the oligomer or the conjugate or the pharmaceutical composition according to the invention to the patient.

The invention provides for a method of lowering the plasma triglyceride in a patient, the method comprising the step of administering the oligomer or the conjugate or the pharmaceutical composition according to the invention to the patient so that the blood serum triglyceride level is reduced.

The invention provides for a method of treating obesity in a patient, the method comprising the step of administering the oligomer or the conjugate or the pharmaceutical composition according to the invention to the patient in need of treatment so that the body weight of the patient is reduced.

The invention provides for a method of treating insulin resistance in a patient, the method comprising the step of administering the oligomer or the conjugate or the pharmaceutical composition according to the invention to the patient in need of treatment so that the patients sensitivity to insulin is increased.

The invention provides for a method of treating type II diabetes in a patient, the method comprising the step of administering the oligomer or the conjugate or the pharmaceutical composition according to the invention to the patient suffering from type II diabetes.

The invention provides for a method for treating a metabolic disorder such as metabolic syndrome, diabetes or atherosclerosis, the method comprising the step of administering the oligomer or the conjugate or the pharmaceutical composition according to the invention to the patient in need thereof.

### BRIEF DESCRIPTION OF THE FIGURES

FIG. 1. Mouse Hepa 1-6 cells were transfected with different oligonucleotides targeting FABP4 using lipofectamine. The FABP4 mRNA expression 24 hours after transfection was measured by qPCR normalized to the house keeping gene GAPDH and presented relative to the Mock control

FIG. 2. Human PC3 cells were transfected with different oligonucleotides targeting FABP4 using Lipofectamine. The FABP4 mRNA expression 24 hours after transfection was measured by qPCR normalized to the house keeping gene GAPDH and presented relative to the Mock control.

FIG. 3: FABP4 oligonucleotides were screened in vitro in mouse macrophage like RAW264.7 cells for potency as due to the known function of FABP4 in macrophages. The cells were transfected using Lipofectamine and analysed 24 hours after transfection. FABP4 mRNA data are presented normalised to GAPDH and relative to mock samples.

FIG. 4: Sequence alignment of the human and mouse FABP4 cDNA sequences. Preferred ‘target’ sequences, SEQ ID NO 5, 6, 7, 8, 9, 10 and 11 are represented in bold.

FIG. 5: Target FABP4 amino acid and polynucleotide sequences from human and mouse.

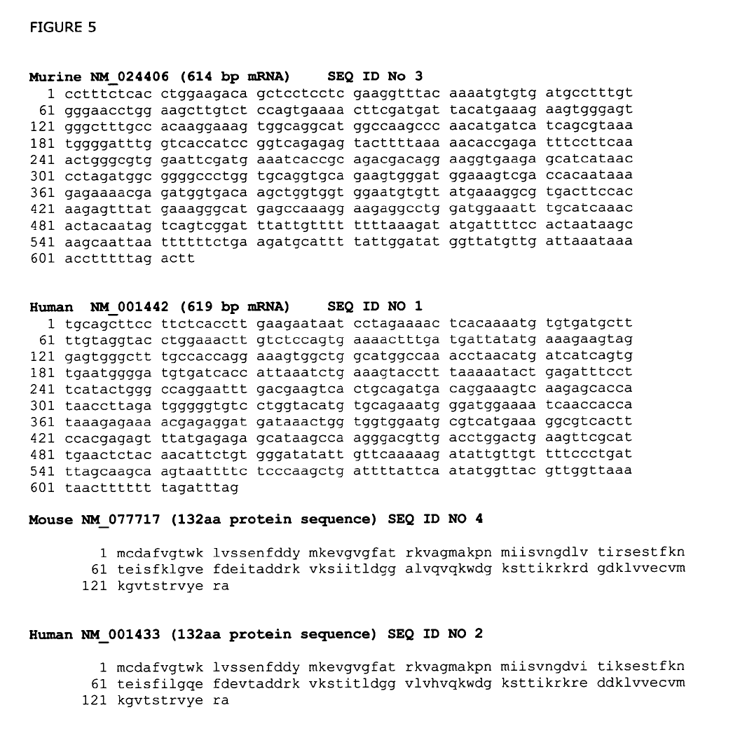

### DETAILED DESCRIPTION OF THE INVENTION

### Oligomers Targeting FABP4

The present invention employs oligomeric compounds (referred herein as oligomers), for use in modulating the function of nucleic acid molecules encoding mammalian FABP4, such as the FABP4 protein shown in SEQ ID NO 2 (human) or SEQ ID NO 4 (mouse), and naturally occurring allelic variants of such nucleic acid molecules encoding mammalian FABP4.

In one embodiment, the oligomer comprises a nucleobase sequence which is at least 80% homologous to a corresponding region of a nucleic acid which encodes a mammalian FABP4.

The oligomer comprises or consists of a contiguous nucleobase sequence.

In one embodiment, the nucleobase sequence of the oligomer consists of the contiguous nucleobase sequence.

However, it is also envisaged that the oligomer may comprise a nucleobase sequence which is at least 80% homologous to a corresponding region of a nucleic acid which encodes a mammalian FABP4, and one or more further nucleobases, such as nucleotides, such as between 1-6 further nucleobases, such as 1, 2, 3, 4, 5 or 6 further nucleobases, which may, for example, be contiguous with either the 5′ most, or 3′ most nucleobase of the contiguous nucleobase sequence. Such further nucleobase or bases may be equivalent to region D as described in the context of a gapmer oligomer herein. In one embodiment one or more of the further nucleobases are nucleotide analogues which stabilise the oligomer in vivo, such as protect the oligomer from nuclease degradation, such as the nucleotide analogues described herein.

The mammalian FABP4 is preferably selected for the group consisting of human or mouse FABP4. Preferably the mammalian FABP4 is human FABP4.

The nucleic acid which encodes the mammalian FABP4 is, in a preferable embodiment, the human FABP4 cDNA sequence is shown as SEQ ID NO 1 and/or the mouse FABP4 cDNA sequence is shown as SEQ ID NO 3, or allelic variants thereof.

The nucleic acid which encodes a mammalian FABP4 may be in the sense or antisense orientation.

It is highly preferable that the oligomer according to the invention is an RNA antagonist, such as an antisense oligonucleotide or siRNA, preferably an antisense oligonucleotide.

Therefore, in a highly preferred embodiment ‘the target’ of the oligomer according to the invention is the FABP4 mRNA. In this embodiment the oligomer may be in the form of an antisense oligonucleotide, or a siRNA, which, when introduced into the cell which is expressing the FABP4 gene, results in reduction of the FABP4 mRNA level, resulting in reduction in the level of expression of the FABP4 in the cell.

The oligomers which target the FABP4 mRNA, may hybridize to any site along the target mRNA nucleic acid, such as the 5′ untranslated leader, exons, introns and 3′ untranslated tail. However, it is preferred that the oligomers which target the FABP4 mRNA hybridise to the mature mRNA form of the target nucleic acid.

When designed as an RNA antagonist, for example, the oligomers of the invention bind to the target nucleic acid and modulate the expression of its cognate protein. Preferably, such modulation produces an inhibition of expression of at least 10% or 20% compared to the normal expression level, more preferably at least a 30%, 40%, 50%, 60%, 70%, 80%, 90% or 95% inhibition compared to the normal expression level. Suitably, such modulation is seen when using between 5 and 25 nM concentrations of the compound of the invention. In the same or a different embodiment, the inhibition of expression is less than 100%, such as less than 98% inhibition, less than 95% inhibition, less than 90% inhibition, less than 80% inhibition, such as less than 70% inhibition. Modulation of expression level is determined by measuring protein levels, e.g. by the methods such as SDS-PAGE followed by western blotting using suitable antibodies raised against the target protein. Alternatively, modulation of expression levels can be determined by measuring levels of mRNA, eg. by northern blotting or quantitative RT-PCR. When measuring via mRNA levels, the level of down-regulation when using an appropriate dosage, such as between 5 and 25 nM concentrations, is, in one embodiment, typically to a level of between 10-20% the normal levels in the absence of the compound of the invention.

It will be recognised that the oligomers of the invention which consists of a contiguous sequence of nucleobases (i.e. nucleobase sequence), may comprise further non-nucleobase components, such as the conjugates herein referred to.

It is recognised that for the production of, for example, a siRNA, the compound of the invention may consist of a duplex of complementary sequence, i.e. a double stranded oligonucleotide, wherein each of the sequences in the duplex is as defined according to a oligomer of the invention. Typically, such siRNAs comprise of 2 complementary short RNA (or equivalent nucleobase units) sequences, such as between 21 and 23 nts long, with, typically a 2 nt 3′ overhang on either end. In order to enhance in vivo update, the siRNAs may be conjugated, such as conjugated to a sterol, such as a cholesterol group (typically at the 3′ or 5′ termini of one or both of the strands). The siRNA may comprise nucleotide analogues such as LNA, as described in WO2005/073378 which is hereby incorporated by reference.

In one aspect of the invention, the oligomer is not essentially double stranded, such as is not an siRNA.

The length of an oligomer (or contiguous nucleobase sequence) will be determined by that which will result in inhibition of the target. For a perfect match with the target, the contiguous nucleotide sequence or oligomer as low as 8 bases may suffice, but it will generally be more, e.g. 10 or 12, and preferably between 12-16. The maximum size of the oligomer will be determined by factors such as cost and convenience of production, ability to manipulate the oligomer and introduce it into a cell bearing the target mRNA, and also the desired binding affinity and target specificity. If too long, it may undesirably tolerate an increased number of mismatches, which may lead to unspecific binding.

In one embodiment, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

Incorporation of affinity-enhancing nucleotide analogues in the oligomer nucleobase sequence, such as LNA or 2′-substituted sugars, preferably LNA, can allow the size of the specifically binding oligonucleotide to be reduced, and may also reduce the upper limit to the size of the oligonucleotide before non-specific or aberrant binding takes place. An affinity enhancing nucleotide analogue is one which, when inserted into the nucleobase sequence of the oligomer results in an increased Tₘ of the oligomer when formed in a duplex with a complementary RNA (such as the mRNA target), as compared to an equivalent oligomer which comprises a DNA nucleotide in place of the affinity enhancing nucleotide analogue.

The oligomer of the invention typically consists or comprises of a contiguous nucleobase sequence of (a total of) between 10 and 50 nucleobases, such as between 10 and 30 nucleobases.

Particularly preferred compounds are oligomers, such as antisense oligonucleotides, comprising of a contiguous nucleobase sequence of from (about) 10 to (about) 30 nucleobases, or from 12 to 25 nucleobases and most preferably are oligomers comprising 13-18 nucleobases such as 14, 15, 16 or 17 nucleobases.

In one embodiment, the oligomer according to the invention consists of no more than 22 nucleobases, such as no more than 20 nucleobases, such as no more than 18 nucleobases, such as 15, 16 or 17 nucleobases, optionally conjugated with one or more non-nucleobase entity, such as a conjugate.

In one embodiment, the oligomer or contiguous nucleobase sequence has a length of between 10-22 nucleobases.

In one embodiment, the oligomer or contiguous nucleobase sequence has a length of between 10-18 nucleobases.

In one embodiment, the oligomer or contiguous nucleobase sequence has a length of between 10-16 nucleobases.

In one embodiment, the oligomer or contiguous nucleobase sequence has a length of between 12-16 nucleobases.

In one embodiment, the oligomer or contiguous nucleobase sequence has a length of between 12-14 nucleobases.

In one embodiment, the oligomer or contiguous nucleobase sequence has a length of between 14-16 nucleobases.

In one embodiment, the oligomer or contiguous nucleobase sequence has a length of between 14-18 nucleobases.

In one embodiment, the oligomer or contiguous nucleobase sequence has a length of 14, 15 or 16 nucleobases.

In one embodiment it is preferred that the oligomer of the invention comprises less than 20 nucleobases.

### Preferred Sequences

Target sequences of the invention may, in one non limiting embodiment, be identified as follows. In a first step conserved regions in the target gene are identified. Amongst those conserved regions, any sequences with polymorphisms are normally excluded (unless required for a specific purpose) as these may affect the binding specificity and/or affinity of an oligomer designed to bind to a target sequence in this region. Any regions with palindromic or repeat sequences are normally excluded. The remaining regions are then analysed and candidate target sequences of suitable length (such as the lengths of the oligomer/contiguous nucleobase sequence referred to herein), e.g. 10-50 nucleobases, preferably 10-25 nucleobases, more preferably 10, 11, 12, 13, 14, 15 or 16 nucleobases are identified. Target sequences which are, based on computer analysis, likely to form structures such as dimers or hairpin structures are normally excluded.

Preferably these candidate target sequences show a high degree of sequence homology throughout the animal kingdom—or at least among animals likely to be required for pre-clinical testing. This allows the use of the identified oligomer sequences, and the corresponding oligomers such as antisense oligonucleotides, to be tested in animal models. Particularly useful are target sequences which are conserved in human, chimpanzee, dog, rat, mouse, and most preferred in human, and mouse (and/or rat).

In one embodiment, the oligomer of the invention may comprise both a polynucleotide region, i.e. a nucleobase region, which typically consists of a contiguous sequence of nucleobases/nucleotides, and a further non-nucleobase region. When referring to the compound of the invention consisting of a nucleobase sequence, the compound may comprise non-nucleobase components, such as a conjugate component.

Alternatively, the oligomer of the invention may consist entirely of a nucleobase region.

In one embodiment the oligomers of the invention may comprise or consist of a (contiguous) nucleobase sequence, such as 12, 13, 14, 15, 16, 17 or 18 contiguous nucleobases, which correspond to a contiguous nucleotide sequence present in a sequence selected from the group consisting of SEQ ID NOs 5, 6, 7, 8, 9, 10 and 11, or complement thereof, wherein said oligomer (or contiguous nucleobase portion thereof) may optionally comprise one, two, or three mismatches against said selected sequence.

Preferred oligomers may comprise or consist of a (contiguous) nucleobase sequence of between 12-18 contiguous nucleobases in length, such as 12, 13, 14, 15, 16, 17 or 18 contiguous nucleobases, which are complementary to a contiguous nucleotide sequence present in a sequence selected from the group consisting of SEQ ID NOs 5, 6, 7, 8, 9, 10 and 11, wherein said oligomer (or contiguous nucleobase portion thereof) may optionally comprise one, two, or three mismatches against said selected sequence.

Other preferred oligomers include a (contiguous) nucleobase sequence, such as a sequence of 14, 15 or 16 contiguous nucleobases in length, which have a nucleobase sequence selected from a sequence from the group consisting of SEQ ID No 5, 6, 7, and 8, or a complement nucleobase sequence thereof, wherein said oligomer (or contiguous nucleobase portion thereof) may optionally comprise one, two, or three mismatches against said selected sequence.

In one embodiment the oligomer (or contiguous nucleobase portion thereof) is selected from, or comprises, one of the sequences selected from the group consisting of SEQ ID NO 12 to SEQ ID NO 116 inclusive, or a sub-sequence of at least 10 contiguous nucleobases thereof, such as 11, 12, 13, 14, 15 or 16 contiguous nucleobases thereof, wherein said oligomer (or contiguous nucleobase portion thereof) may optionally comprise one, two, or three mismatches against said selected sequence.

In one embodiment the oligomer (or nucleobase portion thereof) is selected from, or comprises, one of the sequences selected from the group consisting of: SEQ ID NO 12, 15, 18, 19, 23, 24 and 27, or a sub-sequence of at least 10 contiguous nucleobases thereof, such as 11, 12, 13, 14, 15, or 16 contiguous nucleobases thereof, wherein said oligomer (or contiguous nucleobase portion thereof) may optionally comprise one, two, or three mismatches against said selected sequence.

In one embodiment the oligomer (or nucleobase portion thereof) consists or comprises of a sequence which is, or corresponds to, a sequence selected from the group consisting of: SEQ ID NO 12 to SEQ ID NO 116, or a contiguous sequence of at least 12, 13, 14, 15, or 16 consecutive nucleobases present in said sequence, wherein the nucleotides present in the oligomer may be substituted with a corresponding nucleotide analogue, wherein said oligomer may optionally comprise one, two, or three mismatches against said selected sequence.

In one embodiment the oligomer according to the invention consists or comprises of a nucleobase sequence according to SEQ ID NO 12, such as SEQ ID NO 118.

In one embodiment the oligomer according to the invention consists or comprises of a nucleobase sequence according to SEQ ID NO 15, such as SEQ ID NO 122.

In one embodiment the oligomer according to the invention consists or comprises of a nucleobase sequence according to SEQ ID NO 18, such as SEQ ID NO 119.

In one embodiment the oligomer according to the invention consists or comprises of a nucleobase sequence according to SEQ ID NO 19, such as SEQ ID NO 120.

In one embodiment the oligomer according to the invention consists or comprises of a nucleobase sequence according to SEQ ID NO 23, such as SEQ ID NO 123.

Preferably the oligomer according to the invention consists or comprises of a nucleobase sequence according to SEQ ID NO 24, such as SEQ ID NO 117.

Preferably the oligomer according to the invention consists or comprises of a nucleobase sequence according to SEQ ID NO 27, such as SEQ ID 121.

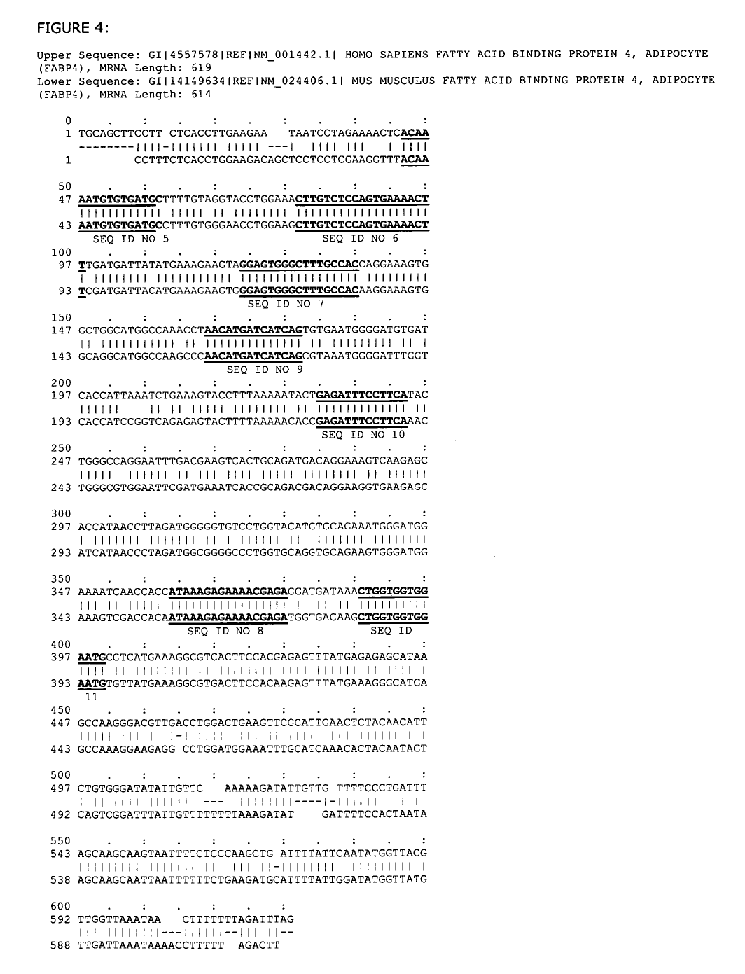

In one embodiment, the contiguous nucleobase sequence of the oligomer of the invention is 100% complementary to at least the human FABP4 target mRNA, and at least one further mammalian FABP4 target, such as the dog, rat, mouse or chimpanzee FAMP4 mRNA/cDNA sequence. It is however envisaged that in such as oligomer, one or two mismatches between the contiguous nucleobase sequence and the human, and/or the other mammalian target may exist, although it is preferred that there are no mismatches. In this respect, FIG. 4 illustrates an alignment between the human and mouse nucleic acids that encode the respective human and mouse FABP4 polypeptides. Table 1 provides suitable FABP4 polynucleotides and the corresponding polypeptides provided by the NCBI Genbank Accession numbers—certain known allelic variants and known homologues from other mammalian species may be easily identified by performing BLAST searches using the sequences referenced in Table 1.

In a preferred embodiment, the oligomer of the invention consists or comprises of a contiguous nucleobase sequence which has at least 12, such as at least 13, such as at least 14, such as at least 15, such as at least 16, such as at least 17, such as at least 18, such as 12, 13, 14, 15, 16, 17 or 18 contiguous nucleobases which are complementary to (the corresponding region) of both the human and or mouse nucleic acids that encode FABP4, such as SEQ ID NO 1 (human) and 3 (mouse)—see FIG. 4.

TABLE 1

| | Nucleic acid (mRNA/cDNA | |
|---|---|---|
| | sequence) | Polypeptide (deduced) |
| Human | NM_001442 (SEQ ID NO 1) | NP_001433 (SEQ ID |
| | | NO 2) |
| Mouse | NM_024406 (SEQ ID NO 3) | NP_077717 (SEQ ID |
| | | NO 4) |
| Rat | NM_053365 | NP_445817 |
| Chimpanzee | XM_519830 | XP_519830 |

### Complementarity and Mismatches

It is preferable that the oligomer comprises a nucleobase sequence which is complementary to the corresponding region of a nucleic acid which encodes a mammalian FABP4—i.e. comprises an antisense nucleobase sequence.

Particularly preferred oligomers are those which consist or comprise of a contiguous nucleobase sequence which is complementary to between 10-30 nucleotides present in the nucleic acids which encode the human and/or mouse FABP4, such as SEQ ID NO 1 and/or 3, or allelic variants thereof.

However, in some embodiments, the oligomer may tolerate 1, 2, 3, 4, or 4 (or more) mismatches, when hybridising to the target sequence and still sufficiently bind to the target to show the desired effect, i.e. down-regulation of the target. Mismatches may, for example, be compensated by increased length of the oligomer nucleobase sequence and/or an increased number of analogues, such as LNA, present within the nucleobase sequence.

In one embodiment, the contiguous nucleobase sequence comprises no more than 3, such as no more than 2 mismatches to the corresponding region of a nucleic acid which encodes a mammalian FABP4.

In one embodiment, the contiguous nucleobase sequence comprises no more than a single mismatch to the corresponding region of a nucleic acid which encodes a mammalian FABP4.

In one embodiment, the oligomer is at least 80% homologous to the complement of a corresponding region of a nucleic acid which encodes a mammalian FABP4, i.e. is at least 80% complementary to the nucleic acid which encodes the mammalian FABP4, such as at least 85%, 90%, 91%, 92%, 93%, 93⅓%, 93.75%, 94%, 95%, 96% or at least 97% complementary, such at least 98% complementary, such as 100% complementary (fully complementary) to the corresponding region of the nucleic acid which encodes the mammalian FABP4.

It is to be understood that where we refer to ‘complementary’ herein, where there is no indication of the percentage complementarity, it is to be understood that we refer to fully complementary—i.e. 100% complementary.

In one embodiment, the oligomer of the invention consists of a contiguous nucleobase sequence with is 100% complementary to a corresponding region of the corresponding sequence present in the nucleic acid which encodes the FABP4 polypeptide, such as SEQ ID NO 1 and/or 3 or naturally occurring allelic variants thereof.

However, it is considered that, in one embodiment, the oligomer or contiguous nucleobase sequence, may comprise one or more mismatches when compared to the nucleic acid which encodes the FABP4 polypeptide.

The oligomer of the invention, preferably, does not comprise more than four, such as not more than three, such as not more than two, such as not more than one mismatch, with the corresponding region of the sequence present in the nucleic acid which encodes the FABP4 polypeptide, such as SEQ ID NO 1 and/or 3, or naturally occurring allelic variants thereof.

### Nucleotide Analogues

It will be recognised that when referring to a preferred nucleotide sequence motif or nucleotide sequence, which consists of only nucleotides, the oligomers of the invention which are defined by that sequence may comprise a corresponding nucleotide analogues in place of one or more of the nucleotides present in said sequence, such as LNA units or other nucleotide analogues, which raise the duplex stability/Tₘ of the oligomer/target duplex (i.e. affinity enhancing nucleotide analogues).

Furthermore, the nucleotide analogues may enhance the stability of the oligomer in vivo.

Examples of suitable and preferred nucleotide analogues are provided by PCT/DK2006/000512 or are referenced therein.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

Suitably, when the nucleobase sequence of the oligomer, or the contiguous nucleobase sequence, is not fully complementary to the corresponding region of the FABP4 target sequence, in one embodiment, when the oligomer comprises affinity enhancing nucleotide analogues, such nucleotide analogues form a complement with their corresponding nucleotide in the FABP4 target.

The oligomer may thus comprise or consist of a simple sequence of natural nucleotides—preferably 2′-deoxynucleotides (referred to here generally as “DNA”), but also possibly ribonucleotides (referred to here generally as “RNA”)—or it could comprise one or more (and possibly consist completely of) nucleotide “analogues”.

Nucleotide “analogues” are variants of natural DNA or RNA nucleotides by virtue of modifications in the sugar and/or base and/or phosphate portions. The term “nucleobase” will be used to encompass natural (DNA- or RNA-type) nucleotides as well as such “analogues” thereof. Analogues could in principle be merely “silent” or “equivalent” to the natural nucleotides in the context of the oligonucleotide, i.e. have no functional effect on the way the oligonucleotide works to inhibit beta-catenin expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell.

Examples of such modification of the nucleotide include modifying the sugar moiety to provide a 2′-substituent group or to produce a bridged (locked nucleic acid) structure which enhances binding affinity and probably also provides some increased nuclease resistance; modifying the internucleotide linkage from its normal phosphodiester to one that is more resistant to nuclease attack, such as phosphorothioate or boranophosphate—these two, being cleavable by RNase H, also allow that route of antisense inhibition in modulating the beta-catenin expression.

A preferred nucleotide analogue is LNA, such as beta-D-oxy-LNA, alpha-L-oxy-LNA, beta-D-amino-LNA and beta-D-thio-LNA, most preferred beta-D-oxy-LNA.

In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

In some embodiments the nucleotide analogues present within the oligomer of the invention in regions A and C mentioned herein are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid) units and 2′MOE units.

2′-O-methoxyethyl-RNA (2′MOE), 2′-fluoro-DNA monomers and LNA are preferred nucleotide analogues, and as such the oligonucleotide of the invention may comprise nucleotide analogues which are independently selected from these three types of analogue, or may comprise only one type of analogue selected from the three types.

Preferably, the oligomer according to the invention comprises at least one Locked Nucleic Acid (LNA) unit, such as 1, 2, 3, 4, 5, 6, 7, or 8 LNA units, preferably between 4 to 8 LNA units, most preferably 4, 5 or 6 LNA units. Suitably, the oligomer may comprise both beta-D-oxy-LNA, and one or more of the following LNA units: thio-LNA, amino-LNA, oxy-LNA, ena-LNA and/or alpha-LNA in either the D-beta or L-alpha configurations or combinations thereof.

In one embodiment of the invention, the oligomer may comprise both LNA and DNA units. Preferably the combined total of LNA and DNA units is 10-25, preferably 10-20, even more preferably 12-16.

In one embodiment of the invention, the nucleobase sequence of the oligomer, such as the contiguous nucleobase sequence consists of at least one LNA and the remaining nucleobase units are DNA units.

In some embodiments of oligomer according to the invention, such as an antisense oligonucleotide which comprises LNA, all LNA C units are 5′ methyl-Cytosine. In some embodiments, all the nucleotide analogues are LNA.

In most preferred embodiments the oligomer comprises only LNA nucleotide analogues and nucleotides (RNA or DNA, most preferably DNA nucleotides, optionally with modified internucleobase linkages such as phosphorothioate).

In some embodiments at least one of said nucleotide analogues is 2′-MOE-RNA, such as 2, 3, 4, 5, 6, 7 or 8 2′-MOE-RNA nucleobase units.

In some embodiments at least one of said nucleotide analogues is 2′-fluoro DNA, such as 2, 3, 4, 5, 6, 7 or 8 2′-fluoro-DNA nucleobase units.

Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1:

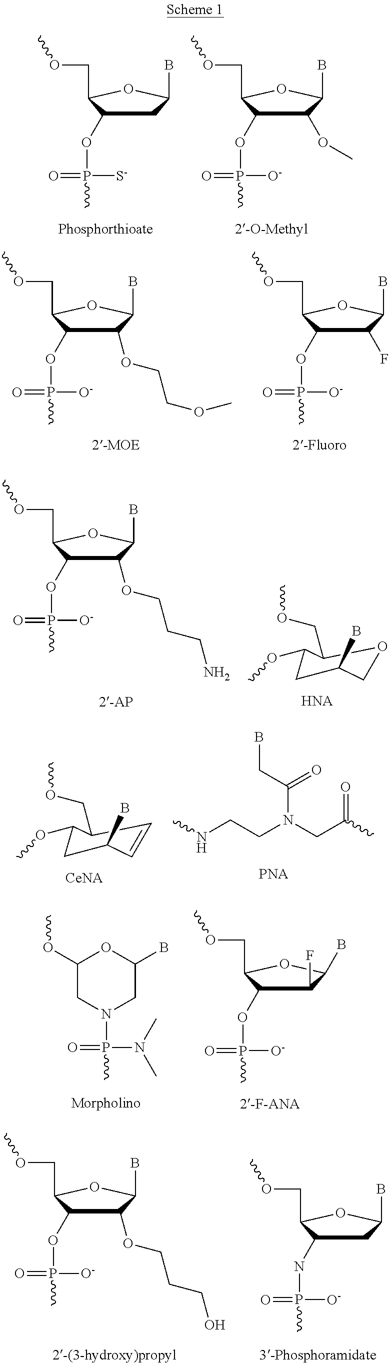

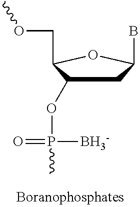

The term “LNA” refers to a bicyclic nucleotide analogue, known as “Locked Nucleic Acid”. It may refer to an LNA monomer, or, when used in the context of an “LNA oligonucleotide” refers to an oligonucleotide containing one or more such bicyclic nucleotide analogues. The LNA used in the oligonucleotide compounds of the invention preferably has the structure of the general formula

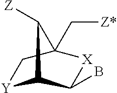

where X and Y are independently selected among the groups —O—, —S—, —N(H)—, N(R)—, —CH₂— or —CH— (if part of a double bond), —CH₂—O—, —CH₂—S—, —CH₂—N(H)—, —CH₂—N(R)—, —CH₂—CH₂— or —CH₂—CH— (if part of a double bond), —CH═CH—, where R is selected from hydrogen and C₁₋₄-alkyl;
Z and Z* are independently selected among an internucleoside linkage, a terminal group or a protecting group;
B constitutes a natural or non-natural nucleotide base moiety; and the asymmetric groups may be found in either orientation.
Preferably, the LNA used in the oligomer of the invention comprises at least one LNA unit according any of the formulas

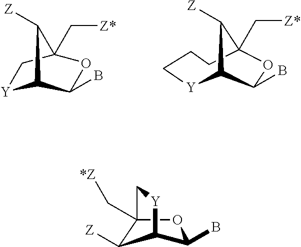

wherein Y is —O—, —S—, —NH—, or N(R^{H}); Z and Z* are independently selected among an internucleoside linkage, a terminal group or a protecting group; B constitutes a natural or non-natural nucleotide base moiety, and R^{H} is selected from hydrogen and C₁₋₄-alkyl.
Preferably, the LNA used in the oligomer of the invention comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected form hydrogen and C₁₋₄-alkyl.
Specifically preferred LNA units are shown in scheme 2:

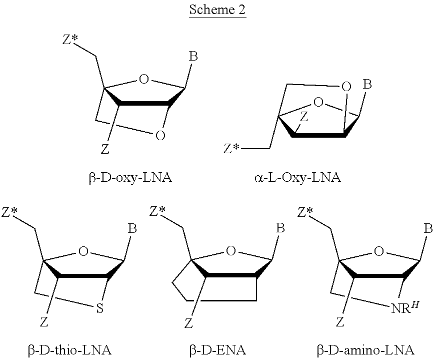

The term “thio-LNA” comprises a locked nucleotide in which at least one of X or Y in the general formula above is selected from S or —CH₂—S—. Thio-LNA can be in both beta-D and alpha-L-configuration.
The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.
The term “oxy-LNA” comprises a locked nucleotide in which at least one of X or Y in the general formula above represents —O— or —CH₂—O—. Oxy-LNA can be in both beta-D and alpha-L-configuration.
The term “ena-LNA” comprises a locked nucleotide in which Y in the general formula above is —CH₂—O— (where the oxygen atom of —CH₂—O— is attached to the 2′-position relative to the base B).
In a preferred embodiment LNA is selected from beta-D-oxy-LNA, alpha-L-oxy-LNA, beta-D-amino-LNA and beta-D-thio-LNA, in particular beta-D-oxy-LNA.
Whenever intervals are described with a the term “between”, such as e.g. “between 3 to 9 nucleotide analogues”, “between 2 and 8 nucleotide analogues” or “between 12-20”, both the first and last number of the described interval are included.
Preferably, the oligomer according to the invention comprises at least one nucleotide analogue, such as Locked Nucleic Acid (LNA) unit, such as 1, 2, 3, 4, 5, 6, 7, 8, 9, or 10 nucleotide analogues, such as Locked Nucleic Acid (LNA) units, preferably between 3 to 9 nucleotide analogues, such as LNA units, such as 4-8, nucleotide analogues, such as LNA units, such as 6-9 nucleotide analogues, such as LNA units, preferably 6, 7 or 8 nucleotide analogues, such as LNA units.
The oligomer according to the invention, such as an antisense oligonucleotide, may comprises of 1, 2, 3, 4, 5, 6, 7, 8, 9, 10, 11, 12, 13, 14 or 15 nucleotide analogues, such as LNA units, in particular 3, 4, 5, 6, 7, 8, 9 or 10 nucleotide analogues, such as LNA units, such as between 1 and 10 nucleotide analogues, such as LNA units such as between 2 and 8 nucleotide analogues such as LNA units.
Preferably the LNA units comprise at least one beta-D-oxy-LNA unit(s) such as 2, 3, 4, 5, 6, 7, 8, 9, or 10 beta-D-oxy-LNA units.
The oligomer of the invention, such as the antisense oligonucleotide, may comprise more than one type of LNA unit. Suitably, the compound may comprise both beta-D-oxy-LNA, and one or more of the following LNA units: thio-LNA, amino-LNA, oxy-LNA, ena-LNA and/or alpha-LNA in either the D-beta or L-alpha configurations or combinations thereof.
Preferably, the oligomer, such as an antisense oligonucleotide, may comprise both nucleotide analogues, such as LNA units, and DNA units. Preferably the combined total of nucleobases, such as, LNA and DNA units, is between 12-20, such as between 14-20, such as 15-18, such as 15, 16 or 17 nucleobase units. Preferably the ratio of nucleotide analogues to DNA present in the oligomeric compound of the invention is between 0.3 and 1, more preferably between 0.4 and 0.9, such as between 0.5 and 0.8.
Benefits of utilising LNA and methods of preparing and purifying LNA and LNA oligonucleotides are disclosed in PCT/DK2006/000512 which are hereby incorporated by reference.
In one embodiment, the oligomer of the invention does not comprise any RNA units.
Nucleotide analogues which increase the Tₘ of the oligomer/target nucleic acid target, as compared to the equivalent nucleotide are preferred (affinity enhancing nucleotide analogues). The oligomers may suitably be capable of hybridising against the target nucleic acid, such as a FABP4 mRNA, to form a duplex with a Tₘ of at least 37° C., such as at least 40° C., at least 50° C., at least 55° C., or at least 60° C. In one aspect, for example, the Tₘ is between 37° C. and 80° C., such as between 50 and 70° C.

### RNAse H Recruitment and Gapmer Oligonucleotides.

It is preferable that the oligomers, or contiguous nucleobase sequence, comprises of a region of at least 6, such as at least 7 consecutive nucleobase units, such as at least 8 or at least 9 consecutive nucleobase units (residues), including 7, 8, 9, 10, 11, 12, 13, 14, 15 or 16 consecutive nucleobases, which, when formed in a duplex with the complementary target FABP4 RNA is capable of recruiting RNaseH. Such regions are referred to as sub-sequences, herein. In one embodiment the sub-sequence is the region B as referred to in the context of a gapmer herein.

EP 1 222 309 provides in vitro methods for determining RNaseH activity, which may be used to determine the ability to recruit RNaseH. A oligomer is deemed capable of recruiting RNase H if, when provided with the complementary RNA target, it has an initial rate, as measured in pmol/l/min, of at least 1%, such as at least 5%, such as at least 10% or less than 20% of the equivalent DNA only oligonucleotide, with no 2′ substitutions, with phosphorothioate linkage groups between all nucleotides in the oligonucleotide, using the methodology provided by Example 91-95 of EP 1 222 309.

An oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphiothioate linkage groups between all nucleotides in the oligonucleotide, using the methodology provided by Example 91-95 of EP 1 222 309.

Typically the region of the oligomer which forms the consecutive nucleobase units which, when formed in a duplex with the complementary target FABP4 RNA is capable of recruiting RNaseH are nucleobase units which form a DNA/RNA like duplex with the RNA target—and include both DNA units and LNA units which are in the alpha-L configuration, particularly preferred being alpha-L-oxy LNA.

The oligomer of the invention may comprise a nucleobase sequence which comprises both nucleotides and nucleotide analogues, and may be in the form of a gapmer, a headmer or a mixmer.

A headmer is defined by a contiguous stretch of nucleotide analogues at the 5′-end followed by a contiguous stretch of DNA or modified nucleobases units recognizable and cleavable by the RNaseH towards the 3′-end (such as at least 7 such nucleobases), and a tailmer is defined by a contiguous stretch of DNA or modified monomers recognizable and cleavable by the RNaseH at the 5′-end (such as at least 7 such nucleobases), followed by a contiguous stretch of nucleotide analogues towards the 3′-end. Other chimeras according to the invention, called mixmers consisting of an alternate composition of DNA or modified monomers recognizable and cleavable by RNaseH and nucleotide analogues. Some nucleotide analogues may also be able to mediate RNaseH binding and cleavage. Since α-L-LNA recruits RNaseH activity to a certain extent, smaller gaps of DNA or modified monomers recognizable and cleavable by the RNaseH for the gapmer construct might be required, and more flexibility in the mixmer construction might be introduced.

Preferably, the oligomer of the invention is an antisense oligonucleotide which is a gapmer.

Preferably the gapmer comprises a (poly)nucleobase sequence of formula (5′ to 3′), A-B-C (and optionally D), wherein;

A (5′ region) consists or comprises of at least one nucleotide analogue, such as at least one LNA unit, such as between 1-6 nucleotide analogues, such as LNA units, preferably between 2-5 nucleotide analogues, such as 2-5 LNA units, such as 2, 3 or 4 nucleotide analogues, such as 2, 3 or 4 LNA units and;

B (central domain), preferably immediately 3′ (i.e. contiguous) to A, consists or comprises of at least five consecutive nucleobases which are capable of recruiting RNaseH, such as between 5-12, such as 5, 6, 7, 8, 9, 10, 11 or 12 consecutive nucleobases which are capable of recruiting RNaseH.

C (3′ region) preferably immediately 3′ to B, consists or comprises at of at least one nucleotide analogues, such as at least one LNA unit, such as between 1-6 nucleotide analogues, such between 2-5 nucleotide analogues, such as between 2-5 LNA units, most preferably 2, 3 or 4 nucleotide analogues, such as 2, 3 or 4 LNA units.

D (optional 3′ terminal), may, where present, consist of one or two nucleotides, such as DNA nucleotides.

Preferred gapmer designs are disclosed in WO2004/046160.

Preferred gapmer designs include, when:
A Consists or comprises of 2, 3 or 4 consecutive nucleotide analogues
B Consists or comprises of 7, 8, 9 or 10 consecutive DNA nucleotides or equivalent nucleobases which are capable of recruiting RNaseH
C Consists or comprises of 2, 3 or 4 consecutive nucleotide analogues
D Consists, where present, of one DNA nucleotide.
Or when
A Consists or comprises of 3 or 4 consecutive nucleotide analogues
B Consists or comprises of 7, 8, 9 or 10 consecutive DNA nucleotides or equivalent nucleobases which are capable of recruiting RNaseH
C Consists or comprises of 3 or 4 consecutive nucleotide analogues
D Consists, where present, of one DNA nucleotide.
Or when
A Consists or comprises of 3 consecutive nucleotide analogues
B Consists or comprises of 9 consecutive DNA nucleotides or equivalent nucleobases which are capable of recruiting RNaseH
C Consists or comprises of 3 consecutive nucleotide analogues
D Consists, where present, of one DNA nucleotide.
In the above embodiment, B may also be selected from the group of sizes of 7, 8, 9, 10, 11, or 12 consecutive DNA nucleotides.
Or when
A Consists or comprises of 4 consecutive nucleotide analogues
B Consists or comprises of 8 consecutive DNA nucleotides or equivalent nucleobases which are capable of recruiting RNaseH
C Consists or comprises of 4 consecutive nucleotide analogues
D Consists, where present, of one DNA nucleotide.

In one embodiment, regions A and/or C consists of the specified number of nucleotide analogues.

It is recognised that in one embodiment that region A and/or C may also comprise of 5′ terminal nucleotide units (not nucleotide analogues)—such as a further 1, 2, 3, or 4 nucleotides—these may be in addition to the specified nucleobases of regions A or C. However, it is preferred that regions A and C consist of the defined nucleobases.

Region B may comprise or consist of DNA units. In one embodiment, region B (central domain), consists or comprises of at least one DNA sugar unit, such as 1-12 DNA units, such as 1, 2, 3, 4, 5, 6, 7, 8, 9 10, 11 or 12 DNA units, preferably between 4-12 DNA units, more preferably between 6-10 DNA units, such as between 7-10 DNA units, most preferably 8, 9 or 10 DNA units.

In one embodiment, which may be the same or different, region B consists of the specified number of nucleobases which are capable of recruiting RNaseH.

One or more of the DNA nucleotides in the central domain (B) may be substituted with one or more nucleotide analogues which are capable of recruiting RNAse H, or even all the DNA nucleotides may be substituted with nucleotide analogues which are capable or recruiting RNAse H. LNA nucleobases which form the alpha-L configuration, such as alpha-L-oxy LNA are particularly preferred nucleotide analogues which may be incorporated into region B as they are capable of recruiting RNaseH. In this respect region B may comprise both alpha-L-LNA and DNA units. Region B may comprise an alpha-L-LNA unit, which may, for example, be at position 1, 2, 3, 4, 5, 6, 7, 8, 9, 10, 11 or 12 of region B (as determined from either the 3′ or 5′ end), and in one embodiment the remaining nucleobases of region B may be DNA, or alternatively region B may comprise one or more further alpha-L-LNA units, such as 2, 3, 4, 5, 6, 7, 8, 9, 10, or 11 further alpha-L-LNA units. In one embodiment, region B comprises 2 alpha-L-LNA units, and the remaining nucleobase units are DNA. In a further embodiment, region B comprises 3 alpha-L-LNA units, and the remaining nucleobase units are DNA. The alpha-L-units may, in one embodiment be positioned at the 5′ and or 3′ positions of region B, and/or in a non terminal position of region B. Where more than one alpha-L-LNA unit is present in region B, region B may comprise a sequence where the alpha-LNA units are either adjacent to each other (i.e. ant least 5′-LNA-LNA-3′) and/or where the alpha LNA units are non-adjacent, i.e. separated by at least 1, such as 1, 2, 3, 4, 5, 6, 7, 8, 9 or 10 alternative nucleobases or nucleotides, such as DNA units.

In the above embodiments referring to gapmer designs, the gap region ‘B’ may, in some embodiment, be 7, 8, 9 or 10, or more consecutive DNA nucleotides or equivalent nucleobases which are capable of recruiting RNaseH.

Regions A and/or C, in one embodiment do not exceed 10 contiguous nucleobases in length.

Regions A and/or C, in one embodiment do not exceed 8 contiguous nucleobases in length.

Regions A and/or C, in one embodiment do not exceed 6 contiguous nucleobases in length.

Region B, in one embodiment does not exceed 20 contiguous nucleobases in length.

Region B, in one embodiment does not exceed 15 contiguous nucleobases in length.

Region B, in one embodiment does not exceed 12 contiguous nucleobases in length.

In a gapmer oligomer, it is preferable that any mismatches are not within the central domain (B) above, or are outside of at least a minimum stretch of 7 continuous nucleobases of the central domain, such as 7, 8 or 9 or 10 continuous nucleobases, which preferably comprises or consists of DNA units, or alpha-L-LNA (e.g. alpha-L-oxy LNA) as described above.

In a gapmer oligomer, it is preferred that any mismatches are located towards the 5′ or 3′ termini of the gapmer. Therefore, it is preferred that in a gapmer oligonucleotide which comprises mismatches with the target mRNA, that such mismatches are located either in 5′ (A) and/or 3′ (C) regions, and/or said mismatches are between the 5′ or 3′ nucleotide unit of said gapmer oligonucleotide and target molecule.

However, in one embodiment, the gap region may comprise a mismatch, such as in a position in the middle or within the middle two or three nucleobases within the gap region (B).

In one embodiment, the gapmer, of formula A-B-C, further comprises a further region, D, which consists or comprises, preferably consists, of one or more DNA sugar residue terminal of the 3′ region (C) of the oligomeric compound, such as between one and three DNA sugar residues, including between 1 and 2 DNA sugar residues, most preferably 1 DNA sugar residue.

In one embodiment, within the compound according to the invention, such as an antisense oligonucleotide, which comprises LNA, all LNA C residues are 5′ methyl-Cytosine.

Preferably the LNA units of the oligomer, such as an antisense oligonucleotide, of the invention are selected from one or more of the following: thio-LNA, amino-LNA, oxy-LNA, ena-LNA and/or alpha-LNA in either the D-beta or L-alpha configurations or combinations thereof. Beta-D-oxy-LNA is a preferred LNA for use in the oligomer of the invention, particularly in regions A and C (where as alpha-L-LNA are preferred, when present, in region B). Thio-LNA may also be preferred for use in the oligomer of the invention. Amino-LNA may also be preferred for use in the oligomer of the invention. Oxy-LNA may also be preferred for use in the oligomer of the invention. Ena-LNA may also be preferred for use in the oligomer of the invention. Alpha-LNA may also be preferred for use in the oligomer of the invention.

### Internucleoside Linkages

Suitable internucleoside linkages include those listed within PCT/DK2006/000512, for example the internucleoside linkages listed on the first paragraph of page 34 of PCT/DK2006/000512 (hereby incorporated by reference).

Suitable sulphur (S) containing internucleoside linkages as provided above may be preferred. Phosphorothioate internucleotide linkages are also preferred, particularly for the gap region (B) of gapmers. Phosphorothioate linkages may also be used for the flanking regions (A and C, and for linking C to D, and D). Regions A, B and C, may however comprise internucleoside linkages other than phosphorothioate, such as phosphodiester linkages, particularly, for instance when the use of nucleotide analogues protects the internucleoside linkages within regions A and C from endo-nuclease degradation—such as when regions A and C comprise LNA nucleobases.

The internucleobase linkages in the oligomer may be phosphodiester, phosphorothioate or boranophosphate so as to allow RNaseH cleavage of targeted RNA. Phosphorothioate is preferred, for improved nuclease resistance and other reasons, such as ease of manufacture.

In one aspect of the oligomer of the invention, the nucleobases (nucleotides and/or nucleotide analogues) are linked to each other by means of phosphorothioate groups.

In some embodiments region A comprises at least one phosphodiester linkage between two nucleotide analogue units, or a nucleotide analogue unit and a nucleobase unit of Region B. In some embodiments region C comprises at least one phosphodiester linkage between two nucleotide analogue units, or a nucleotide analogue unit and a nucleobase unit of Region B.

In some embodiments, region C comprises at least one phosphodiester linkage between a nucleotide analogue unit and a nucleobase unit of Region D.

In some embodiments the internucleobase linkage between the 3′ nucleotide analogue of region A and the 5′ nucleobase of region B is a phosphodiester.

In some embodiments the internucleobase linkage between the 3′ nucleobase of region B and the 5′ nucleotide analogue of region C is a phosphodiester.

In some embodiments the internucleobase linkage between the two adjacent nucleotide analogues at the 5′ end of region A are phosphodiester.

In some embodiments the internucleobase linkage between the two adjacent nucleotide analogues at the 3′ end of region C is phosphodiester.

In some embodiments the internucleobase linkage between the two adjacent nucleotide analogues at the 3′ end of region A is phosphodiester.

In some embodiments the internucleobase linkage between the two adjacent nucleotide analogues at the 5′ end of region C is phosphodiester.

In some embodiments region A has a length of 4 nucleotide analogues and the internucleobase linkage between the two middle nucleotide analogues of region A is phosphodiester.

In some embodiments region C has a length of 4 nucleotide analogues and internucleobase linkage between the two middle nucleotide analogues of region C is phosphodiester.

In some embodiments all the internucleobase linkages between nucleotide analogues present in the compound of the invention are phosphodiester.

In some embodiments, such as the embodiments referred to above, where suitable and not specifically indicated, all remaining internucleobase linkages are either phosphodiester or phosphorothioate, or a mixture thereof.

In some embodiments all the internucleobase linkage groups are phosphorothioate.

When referring to specific gapmer oligonucleotide sequences, such as those provided herein it will be understood that, in one embodiment, when the linkages are phosphorothioate linkages, alternative linkages, such as those disclosed herein may be used, for example phosphate (phosphodiester) linkages may be used, particularly for linkages between nucleotide analogues, such as LNA, units. Likewise, when referring to specific gapmer oligonucleotide sequences, such as those provided herein, when the C residues are annotated as 5′ methyl modified cytosine, in one embodiment, one or more of the Cs present in the oligonucleotide may be unmodified C residues.

### Method of Identification and Preparation of Compounds of the Invention:

The oligomers of the invention, which modulate expression of the target, may be identified through experimentation or though rational design based on sequence information on the target and know-how on how best to design an oligomeric compound against a desired target. The sequences of these compounds are preferred embodiments of the invention. Likewise, the sequence motifs in the target to which these preferred oligomeric compounds are complementary (referred to as “hot spots”) are preferred sites for targeting.

In many cases the identification of an oligomer, such as an LNA oligonucleotide, effective in modulating FABP4 expression or activity in vivo or clinically is based on sequence information on the target gene (such as SEQ ID NO 1 and/or 3). However, one of ordinary skill in the art will appreciate that such oligomeric compounds can also be identified by empirical testing. oligomeric compounds having, for example, less sequence homology, greater or fewer modified nucleotides, or longer or shorter lengths, compared to those of the preferred embodiments, but which nevertheless demonstrate responses in clinical treatments, are also within the scope of the invention.

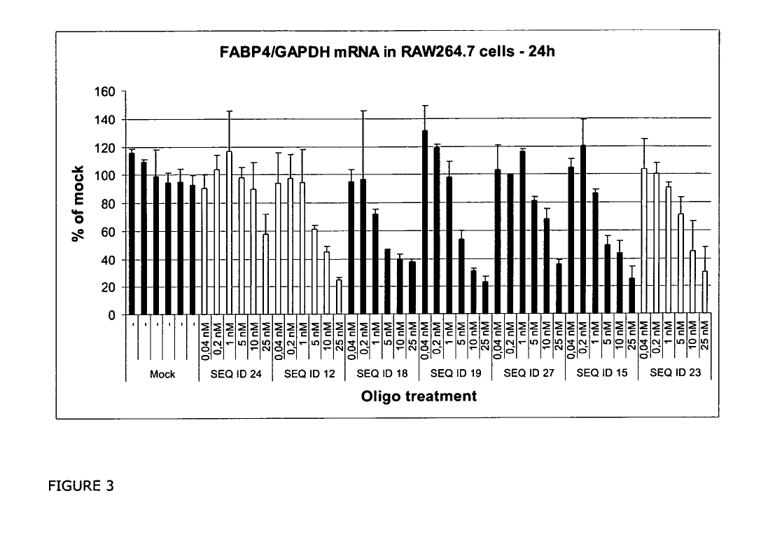

Amino acid and polynucleotide homology may be determined using ClustalW algorithm using standard settings: see http://www.ebi.ac.uk/emboss/align/index.html, Method: EMBOSS::water (local): Gap Open=10.0, Gap extend=0.5, using Blosum 62 (protein), or DNAfull for nucleotide sequences. As illustrated in FIG. 3, such alignments can also be used to identify regions of the nucleic acids encoding FABP4 from human and a different mammalian species, such as monkey, mouse and/or rat, where there are sufficient stretches of nucleic acid complementarily to allow the design of oligonucleotides which target both the human FABP4 target nucleic acid, and the corresponding nucleic acids present in the different mammalian species, such as regions of at least 10, such as at least 12, such as at least 14, such as at least 16, such as at least 18, such as 12, 13, 14, 15, 16, 17 or 18 contiguous nucleobases which are 100% complementary to both the nucleic acid encoding FABP4 from humans and the nucleic acid(s) encoding FABP4 from the different mammalian species.

### DEFINITIONS

When determining “homology” between the oligomers of the invention (or contiguous nucleobase sequence) and the nucleic acid which encodes the mammalian FABP4, such as those disclosed herein, the determination of homology may be made by a simple alignment with the corresponding nucleobase sequence of the compound of the invention and the corresponding region of the nucleic acid which encodes the mammalian FABP4 (or target nucleic acid), and the homology is determined by counting the number of bases which align and dividing by the total number of contiguous bases in the compound of the invention, and multiplying by 100. In such a comparison, if gaps exist, it is preferable that such gaps are merely mismatches rather than areas where the number of nucleobases within the gap differs between the nucleobase sequence of the invention and the target nucleic acid.

The terms “located within” and “corresponding to”/“corresponds to” refer to the comparison between the nucleobase sequence of the oligomer or contiguous nucleobase sequence and the equivalent nucleotide sequence of i) the reverse complement of the nucleic acid target, such as the mRNA which encodes the FABP4 target protein, such as SEQ ID NO 1 or SEQ ID NO 3, and/or ii) the sequence of nucleotides provided in the group consisting of SEQ ID NOS: 5, 6, 7, 8, 9, 10 or 11, or in one embodiment the reverse compliments thereof. Nucleotide analogues are compared directly to their equivalent or corresponding nucleotides.

The terms “corresponding nucleotide analogue” and “corresponding nucleotide” are intended to indicate that the nucleobase in the nucleotide analogue and the nucleotide are identical. For example, when the 2-deoxyribose unit of the nucleotide is linked to an adenine, the “corresponding nucleotide analogue” contains a pentose unit (different from 2-deoxyribose) linked to an adenine.

The term “nucleobase” is used as a collective term which encompasses both nucleotides and nucleotide analogues. A nucleobase sequence is a sequence which comprises at least two nucleotides or nucleotide analogues. In one embodiment the nucleobase sequence may comprise of only nucleotides, such as DNA units, in an alternative embodiment, the nucleobase sequence may comprise of only nucleotide analogues, such as LNA units.

The term “nucleic acid” is defined as a molecule formed by covalent linkage of two or more nucleotides.

The terms “nucleic acid” and “polynucleotide” are used interchangeable herein.

The term “target nucleic acid”, as used herein refers to the DNA encoding mammalian FABP4 polypeptide, such as human FABP4, such as SEQ ID NO1, and/or the mouse (SEQ ID NO 3), rat (Table 1), chimpanzee (Table 1) FABP4 encoding nucleic acids or naturally occurring variants thereof, and RNA nucleic acids derived therefrom, preferably mRNA, such as pre-mRNA, although preferably mature mRNA. In one embodiment, for example when used in research or diagnostics the “target nucleic acid” may be a cDNA or a synthetic oligonucleotide derived from the above DNA or RNA nucleic acid targets. The oligomeric compound according to the invention is preferably capable of hybridising to the target nucleic acid.

The term “naturally occurring variant thereof” refers to variants of the FABP4 polypeptide of nucleic acid sequence which exist naturally within the defined taxonomic group, such as mammalian, such as mouse, rat, monkey, chimpanzee and preferably human. Typically, when referring to “naturally occurring variants” of a polynucleotide the term also may encompasses variants of the FABP4 encoding genomic DNA which are found at the Chromosome 8 Location e.g. by chromosomal translocation or duplication, and the RNA, such as mRNA derived therefrom. When referenced to a specific polypeptide sequence, e.g. SEQ ID NO2 or 4, the term also includes naturally occurring forms of the protein which may therefore be processed, e.g. by co- or post-translational modifications, such as signal peptide cleavage, proteolytic cleavage, glycosylation, etc.

It is preferred that the compound according to the invention is a linear molecule or is synthesised as a linear molecule.

The term “linkage group” is intended to mean a group capable of covalently coupling together two nucleotides, two nucleotide analogues, and a nucleotide and a nucleotide analogue, etc. Specific and preferred examples include phosphate groups and phosphorothioate groups.

In the present context the term “conjugate” is intended to indicate a heterogeneous molecule formed by the covalent attachment of a compound as described herein (i.e. a compound comprising a sequence of nucleotides analogues) to one or more non-nucleotide/non-nucleotide-analogue, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol. When the compound of the invention consists of a nucleobase sequence, it may, in one embodiment further comprise a non-nucleobase portion, such as the above conjugates.

The term “at least one” comprises the integers larger than or equal to 1, such as 1, 2, 3, 4, 5, 6, 7, 8, 9, 10, 11, 12, 13, 14, 15, 16, 17, 18, 19, 20 and so forth.

In one embodiment, such as when referring to the nucleic acid or protein targets of the compounds of the invention, the term “at least one” includes the terms “at least two” and at “least three” and “at least four”, likewise the term “at least two” may comprise the terms at “least three” and “at least four”.

As used herein, the term “pharmaceutically acceptable salts” refers to salts that retain the desired biological activity of the herein identified compounds and exhibit minimal undesired toxicological effects. Non-limiting examples of such salts can be formed with organic amino acid and base addition salts formed with metal cat ions such as zinc, calcium, bismuth, barium, magnesium, aluminium, copper, cobalt, nickel, cadmium, sodium, potassium, and the like, or with a cat ion formed from ammonia, /V,/V-dibenzylethylene-diamine, D-glucosamine, tetraethylammonium, or ethylenediamine; or (c) combinations of (a) and (b); e.g., a zinc tannate salt or the like.

In the present context, the term “C1-4-alkyl” is intended to mean a linear or branched saturated hydrocarbon chain wherein the chain has from one to four carbon atoms, such as methyl, ethyl, n-propyl, isopropyl, n-butyl, isobutyl, sec-butyl and tert-butyl.

As used herein, the term “gene” means the gene including exons, introns, non-coding 5′ and 3′ regions and regulatory elements and all currently known variants thereof and any further variants, which may be elucidated.

As used herein, the terms “RNA antagonist” refers to an oligonucleotide which targets any form of RNA (including pre-mRNA, mRNA, miRNA, siRNA etc).

The term “related disorders” when referring to hypercholesterolemia refers to one or more of the conditions selected from the group consisting of: atherosclerosis, hyperlipidemia, HDL/LDL cholesterol imbalance, primary and secondary dyslipidemias, e.g., familial combined hyperlipidemia (FCHL), acquired hyperlipidemia, statin-resistant hypercholesterolemia, coronary artery disease (CAD), and coronary heart disease (CHD).

In one embodiment, the terms “oligomeric compound” or “oligomer”, which are used interchangeably, refer to an oligonucleotide (which may comprise nucleotides and nucleotide analogues) which can induce a desired therapeutic effect in humans through for example binding by hydrogen bonding to a target nucleic acid. It is also envisaged that the oligomeric compounds disclosed herein may have non-therapeutic applications, such as diagnostic applications. In one embodiment the term “oligomer” may refer to either a single stranded (e.g. antisense oligonucleotide) or a double stranded (e.g. siRNA) oligonucleotide (which may be optionally conjugated to a non-nucleobases entity as described herein). In a preferable embodiment the term “oligomer” refers to a single stranded antisense oligonucleotide.

As used herein, the term “modulation” means either an increase (stimulation) or a decrease (inhibition) in the expression of a gene. In the present invention, inhibition is the preferred form of modulation of gene expression and mRNA is a preferred target—i.e. results in reduction in gene expression.

As used herein, “hybridisation” means hydrogen bonding, which may be Watson-Crick, Holstein, reversed Holstein hydrogen bonding, etc. between complementary nucleotide bases. Watson and Crick showed approximately fifty years ago that deoxyribo nucleic acid (DNA) is composed of two strands which are held together in a helical configuration by hydrogen bonds formed between opposing complementary nucleobases in the two strands. The four nucleobases, commonly found in DNA are guanine (G), adenine (A), thymine (T) and cytosine (C) of which the G nucleobase pairs with C, and the A nucleobase pairs with T. In RNA the nucleobase thymine is replaced by the nucleobase uracil (U), which similarly to the T nucleobase pairs with A. The chemical groups in the nucleobases that participate in standard duplex formation constitute the Watson-Crick face. Hoogsteen showed a couple of years later that the purine nucleobases (G and A) in addition to their Watson-Crick face have a Hoogsteen face that can be recognised from the outside of a duplex, and used to bind pyrimidine oligonucleotides via hydrogen bonding, thereby forming a triple helix structure.

It is highly preferred that the compounds of the invention are capable of hybridizing to the target nucleic acid, such as the mRNA.

### Measurement of Tₘ

A 3 μM solution of the compound in 10 mM sodium phosphate/100 mM NaCl/0.1 nM EDTA, pH 7.0 is mixed with its complement DNA or RNA oligonucleotide at 3 μM concentration in 10 mM sodium phosphate/100 mM NaCl/0.1 nM EDTA, pH 7.0 at 90° C. for a minute and allowed to cool down to room temperature. The melting curve of the duplex is then determined by measuring the absorbance at 260 nm with a heating rate of 1° C./min. in the range of 25 to 95° C. The Tₘ is measured as the maximum of the first derivative of the melting curve.

### Conjugates

In one embodiment of the invention the oligomeric compound is linked to ligands/conjugates, which may be used, e.g. to increase the cellular uptake of antisense oligonucleotides. PCT/DK2006/000512 provides suitable ligands and conjugates, which are hereby incorporated by reference.

The invention also provides for a conjugate comprising the compound according to the invention as herein described, and at least one non-nucleotide or non-polynucleotide moiety covalently attached to said compound. Therefore, in one embodiment where the compound of the invention consists of a specified nucleic acid, as herein disclosed, the compound may also comprise at least one non-nucleotide or non-polynucleotide moiety (e.g. not comprising one or more nucleotides or nucleotide analogues) covalently attached to said compound.

### Applications

The oligomers of the invention may be utilized as research reagents for, for example, diagnostics, therapeutics and prophylaxis.

In research, such oligomers may be used to specifically inhibit the synthesis of FABP4 protein (typically by degrading or inhibiting the mRNA and thereby prevent protein formation) in cells and experimental animals thereby facilitating functional analysis of the target or an appraisal of its usefulness as a target for therapeutic intervention.

In diagnostics the oligomers may be used to detect and quantitate FABP4 expression in cell and tissues by Northern blotting, in-situ hybridisation or similar techniques.

For therapeutics, an animal or a human, suspected of having a disease or disorder, which can be treated by modulating the expression of FABP4 is treated by administering antisense compounds in accordance with this invention. Further provided are methods of treating an animal particular mouse and rat and treating a human, suspected of having or being prone to a disease or condition, associated with expression of FABP4 by administering a therapeutically or prophylactically effective amount of one or more of the oligomers or compositions of the invention.

The pharmaceutical composition according to the invention may be used for the treatment of conditions associated with abnormal levels of FABP4, such as atherosclerosis, diabetes (particularly type II diabetes), and metabolic syndrome.

The pharmaceutical composition according to the invention may be used for the treatment of Alzheimer's disease.

The pharmaceutical composition according to the invention may be used for the treatment of inflammatory diseases, such as arthritis or asthma,

Suitable dosages, formulations, administration routes, compositions, dosage forms, combinations with other therapeutic agents, pro-drug formulations are also provided in PCT/DK2006/000512—which are hereby incorporated by reference, although it should be recognised that the aspects of PCT/DK2006/000512 which are only specifically applicable to the treatment of cancer may not be appropriate in the therapeutic/pharmaceutical compositions and methods of the present invention.

The invention also provides for a pharmaceutical composition comprising a compound or a conjugate as herein described or a conjugate, and a pharmaceutically acceptable diluent, carrier or adjuvant. PCT/DK2006/000512 provides suitable and preferred pharmaceutically acceptable diluent, carrier and adjuvants—which are hereby incorporated by reference.

Pharmaceutical Compositions Comprising More than One Active Ingredient
The pharmaceutical composition according to the invention may further comprise other active ingredients, including those which are indicated as being useful for the treatment of hypercholesterolemia and/or related disorders.
One such class of compounds are statins. The statins are HMG-CoA reductase inhibitors that form a class of hypolipidemic agents, used as pharmaceuticals to lower cholesterol levels in people at risk for cardiovascular disease because of hypercholesterolemia. They work by inhibiting the enzyme HMG-CoA reductase, the enzyme that determines the speed of cholesterol synthesis. Inhibition of this enzyme in the liver stimulates the LDL-receptors, which results in an increased clearance of LDL from the bloodstream and a decrease in blood cholesterol levels. Examples of statins include Atorvastatin™, Cerivastatin™, Fluvastatin™, Lovastatin™, Mevastatin™, Pitavastatin™, Pravastatin™, Rosuvastatin™, and Simvastatin™. The combined use of the compound of the invention and the statins may allow for a reduction in the dose of the statins, therefore overcoming side effects associated with usual dosage of statins, which include, for example, myalgias, muscle cramps, gastrointestinal symptoms, liver enzyme derangements, myositis, myopathy, rhabdomyolysis (the pathological breakdown of skeletal muscle) which may lead to acute renal failure when muscle breakdown products damage the kidney.
Fibrates, a class of amphipathic carboxylic acids is an alternative class of compound which are often combined with statin use, despite an increased frequency of rhabdomyolysis which has been reported with the combined use of statins and fribrates. The composition according to the invention may therefore further comprise fibrates, and optionally statins.
The composition according to the invention may further comprise modulators of Apolipoprotein B (Apo-B), particularly agents which are capable of lowering the expression of function of Apo-B. Suitably, the Apo-B modulators may be antisense oligonucleotides, such as those disclosed in WO 00/97662, WO 03/11887 and WO 2004/44181. A preferred combination is with ISIS compound 301012 (illustrated as SEQ ID NO 13). Further preferred Apo-B modulators are disclosed in U.S. provisional application 60/896,419, hereby incorporated by reference. The composition according to the invention may further comprise modulators of PSCK9 expression, such as antisense oligonucleotides which target PSCK9, the composition may be used in concurrent down-regulation of both FABP4 and PSCK9 expression, resulting in a synergistic effect in terms of blood serum cholesterol and hence advantages when treating hypercholesterolemia and/or related disorders. Such compositions comprising both the compounds of the invention and PSCK9 modulators, such as the antisense oligonucleotides referred to herein, may also further comprise statins. U.S. provisional application 60/828,735, hereby incorporated by reference discloses suitable PCSK9 modulators.
It is also envisaged that the composition may comprise antisense oligonucleotides which comprise nucleotide analogues, such as those disclosed in PCT/DK2006/000481, which is hereby incorporated by reference. Specific LNA oligonucleotides, as disclosed or highlighted are preferred in PCT/DK2006/000481 are especially suited for use in the pharmaceutical composition according to the present invention.
The oligomers of the invention may be combined with fibrates or thiazolidinediones (TZD), for the treatment of diabetes. TZDs are commonly used anti-diabetes drug. Fibrates and TZDs act through PPAR activation, and FABP4 inhibits that action. Therefore oligomers targeting FABP4 are expected to enhance the effect of fibrates and/or TZDs, putatively resulting in need for lower doses of the two drugs.
The invention also provides a kit of parts wherein a first part comprises the oligomer, the conjugate and/or the pharmaceutical composition according to the invention and a further part comprises an antisense oligonucleotide capable of lowering the expression of Apo-B and/or PCSK9. It is therefore envisaged that the kit of parts may be used in a method of treatment, as referred to herein, where the method comprises administering both the first part and the further part, either simultaneously or one after the other.

### Medical Methods and Use

The oligomers and other compositions according to the invention can be used for the treatment of conditions associated with obesity and the metabolic syndrome. It has been suggested by leading scientists in the field that pharmaceutical intervention with FABP4 will result in therapeutic options against obesity, insulin resistance, type 2 diabetes, atherosclerosis, and possibly inflammatory conditions such as arthritis, asthma, or Alzheimer's disease (Makowski and Hotamisligil 2004).

Further conditions which may be associated with abnormal levels of FABP4, and which, therefore may be treated using the compositions, conjugates and compounds according to the invention include disorders selected form the group consisting of: Hyperlipidemias and hyperlipoproteinemias: primary hyperlipoproteinaemia, familial hyperchylomicronemia, familial decreased lipoprotein lipase activity; polygenic hypercholesterolaemia, familial low density lipoprotein receptor deficiency; familial combined hyperlipidemia, familial decreased low density lipoprotein receptor activity; familial dysbetalipoproteinemia, familial defect apolipoprotein E synthesis; Endogenous Hyperlipemia, increased very low density lipoprotein production, decreased very low density lipoprotein clearance; familial hypertriglyceridemia; the metabolic syndrome, syndrome X, pre-diabetes, insulin resistance, type 2 diabetes; cardiovascular disorders including atherosclerosis and coronary artery disease; thrombosis; peripheral vascular disease, and obesity.

Further conditions which may be associated with abnormal levels of FABP4, and which, therefore may be treated using the compositions, conjugates and compounds according to The invention include disorders selected form the group consisting of: von Gierke's disease (glycogen storage disease, type I); lipodystrophies (congenital and acquired forms); Cushing's syndrome; isolated growth hormone deficiency; diabetes mellitus; hyperthyroidism; hypertension; anorexia nervosa; Werner's syndrome; acute intermittent porphyria; primary biliary cirrhosis; extrahepatic biliary 5 obstruction; acute hepatitis; hepatoma; systemic lupus erythematosis; monoclonal gammopathies (including myeloma, multiple myeloma, macroglobulinemia, and lymphoma); endocrinopathies; obesity; nephrotic syndrome; metabolic syndrome; inflammation; Rhematoid arthritis; hypothyroidism; uremia (hyperurecemia); impotence; obstructive liver disease; idiopathic hypercalcemia; dysqlobulinemia; elevated insulin levels; Dupuytren's contracture; AIDS; and Alzheimer's disease and dementia.

The invention further provides methods of preventing cholesterol particle binding to vascular endothelium comprising the step of administering to an individual an amount of a compound of the invention sufficient to FABP4 expression, and as a result, the invention also provides methods of reducing the risk of: (i) cholesterol particle oxidization; (ii) monocyte binding to vascular endothelium; (iii) monocyte differentiation into macrophage; (iv) macrophage ingestion of oxidized lipoprotein particles and release of cytokines (including, but not limited to IL-1, TNF-alpha, TGF-beta); (v) platelet formation of fibrous fibrofatty lesions and inflammation; (vi) endothelium lesions leading to clots; and (vii) clots leading to myocardial infarction or stroke, also comprising the step of administering to an individual an amount of a compound of the invention sufficient to inhibit FABP4 expression.

The therapeutic methods of the invention may also be used for decreasing atherosclerotic plaque formation and methods of increased insulin sensitivity (i.e. decreased insulin resistance).

The invention further provides use of a compound of the invention in the manufacture of a medicament for the treatment of any and all conditions disclosed herein.

Generally stated, one aspect of the invention is directed to a method of treating a mammal suffering from or susceptible to conditions associated with abnormal levels of FABP4, comprising administering to the mammal and therapeutically effective amount of an oligomer targeted to FABP4 that comprises one or more LNA units.

An interesting aspect of the invention is directed to the use of an oligomer (compound) as defined herein or as conjugate as defined herein for the preparation of a medicament for the treatment of a condition according to above.

The methods of the invention are preferably employed for treatment or prophylaxis against diseases caused by abnormal levels of FABP4.

Furthermore, the invention described herein encompasses a method of preventing or treating a disease comprising a therapeutically effective amount of a FABP4 modulating oligomer to a human in need of such therapy. The invention further encompasses the use of a short period of administration of a FABP4 modulating oligonucleotide compound.

In one embodiment of the invention the oligomer (compound) is linked to a ligand or conjugate. For example in order to increase the cellular uptake of the oligomer. In one embodiment the conjugate is a sterols, such as cholesterol.

The oligomers of the invention may also be conjugated to active drug substances, for example, aspirin, ibuprofen, a sulfa drug, an antidiabetic, an antibacterial or an antibiotic.

Alternatively stated, the invention is furthermore directed to a method for treating abnormal levels of FABP4, said method comprising administering a oligomer of the invention, or a conjugate of the invention or a pharmaceutical composition of the invention to a patient in need thereof and further comprising the administration of a further chemotherapeutic agent. Said further administration may be such that the further chemotherapeutic agent is conjugated to the compound of the invention, is present in the pharmaceutical composition, or is administered in a separate formulation.

The invention also relates to an oligomer, a composition or a conjugate as defined herein for use as a medicament.

The invention further relates to use of a compound, composition, or a conjugate as defined herein for the manufacture of a medicament for the treatment of abnormal levels of FABP4. Typically, said abnormal levels of FABP4 is in the form of, or causes, or is characterised by, hypercholesterolemia and related disorders, such as atherosclerosis or hyperlipidemia.

Moreover, the invention relates to a method of treating a subject suffering from a disease or condition selected from hypercholesterolemia and related disorders, such as atherosclerosis, type 2 diabetes, and hyperlipidemia, the method comprising the step of administering a pharmaceutical composition as defined herein to the subject in need thereof. Preferably, the pharmaceutical composition is administered orally.

Examples of related diseases also include different types of HDL/LDL cholesterol imbalance; dyslipidemias, e.g., familial combined hyperlipidemia (FCHL), acquired hyperlipidemia, statin-resistant hypercholesterolemia; coronary artery disease (CAD) coronary heart disease (CHD), atherosclerosis.

It is recognised that when the composition according to the invention also comprises modulators of Apo-B100 expression, such as antisense oligonucleotides which target ApoB-100, the composition may be used in concurrent down-regulation of both FABP4 and ApoB-100 expression, resulting in a synergistic effect in terms of blood serum cholesterol and hence advantages when treating hypercholesterolemia and/or related disorders. Such compositions comprising both the compounds of the invention and ApoB modulators, such as the antisense oligonucleotides referred to herein, may also further comprise statins.

It is recognised that when the composition according to the invention also comprises modulators of PSCK9 expression, such as antisense oligonucleotides which target PSCK9, the composition may be used in concurrent down-regulation of both FABP4 and PSCK9 expression, resulting in a synergistic effect in terms of blood serum cholesterol and hence advantages when treating hypercholesterolemia and/or related disorders. Such compositions comprising both the compounds of the invention and PSCK9 modulators, such as the antisense oligonucleotides referred to herein, may also further comprise statins. U.S. provisional application 60/828,735, hereby incorporated by reference discloses suitable PCSK9 modulators.

### REFERENCES

Fu, Y., Luo, L., Luo, N., & Garvey, W. T. (2006) Lipid metabolism mediated by adipocyte lipid binding protein (ALBP/aP2) gene expression in human THP-1 macrophages. Atherosclerosis 188: 102-111.
Helledie, T., Antonius, M., Sorensen, R. V., Hertzel, A. V., Bernlohr, D. A., Kolvraa, S., Kristiansen, K., & Mandrup, S. (2000) Lipid-binding proteins modulate ligand-dependent trans-activation by peroxisome proliferator-activated receptors and localize to the nucleus as well as the cytoplasm. J Lipid Res 41: 1740-1751.
Hertzel, A. V. & Bernlohr, D. A. (2000) The mammalian fatty acid-binding protein multigene family: molecular and genetic insights into function. Trends Endocrinol. Metab 11: 175-180.
Hotamisligil, G. S., Johnson, R. S., Distel, R. J., Ellis, R., Papaioannou, V. E., & Spiegelman, B. M. (1996) Uncoupling of obesity from insulin resistance through a targeted mutation in aP2, the adipocyte fatty acid binding protein. Science 274: 1377-1379.
Kazemi, M. R., McDonald, C. M., Shigenaga, J. K., Grunfeld, C., & Feingold, K. R. (2005) Adipocyte fatty acid-binding protein expression and lipid accumulation are increased during activation of murine macrophages by toll-like receptor agonists. Arterioscler. Thromb. Vasc. Biol 25: 1220-1224.
Makowski, L., Boord, J. B., Maeda, K., Babaev, V. R., Uysal, K. T., Morgan, M. A., Parker, R. A., Suttles, J., Fazio, S., Hotamisligil, G. S., & Linton, M. F. (2001) Lack of macrophage fatty-acid-binding protein aP2 protects mice deficient in apolipoprotein E against atherosclerosis. Nat. Med. 7: 699-705.
Makowski, L. & Hotamisligil, G. S. (2004) Fatty acid binding proteins—the evolutionary crossroads of inflammatory and metabolic responses. J Nutr. 134: 2464S-2468S.
Tuncman, G., Erbay, E., Hom, X., De, V., I, Campos, H., Rimm, E. B., & Hotamisligil, G. S. (2006) A genetic variant at the fatty acid-binding protein aP2 locus reduces the risk for hypertriglyceridemia, type 2 diabetes, and cardiovascular disease. Proc Natl Acad Sci USA 103: 6970-6975.

### EXAMPLES

### Targets

### Mouse:

Official Symbol: Fabp4 and Name: fatty acid binding protein 4, adipocyte [Mus musculus]
Other Aliases: ALBP/Ap2, Ap2, Lbpl
SEQ ID NO 3

### Human:

Official Symbol: FABP4 and Name: fatty acid binding protein 4, adipocyte [Homo sapiens]
Other Aliases: A-FABP
SEQ ID NO 1
   Fatty acid binding protein 4 (FABP4) also called aP2 (adipocyte fatty acid binding protein) is expressed predominantly in adipocytes and macrophages and plays an important role in diet induced obesity, atherosclerosis and insulin resistance. FABP4 is a cytoplasmic protein that is transcriptionally regulated by fatty acids. It is thought to be involved in fatty acid uptake, transport and metabolism.
   The effect of antisense compounds on target nucleic acid expression can be tested in any of a variety of cell types provided that the target nucleic acid is present at measurable levels. Target can be expressed endogenously or by transient or stable transfection of a nucleic acid encoding said nucleic acid.
   The expression level of target nucleic acid can be routinely determined using, for example, Northern blot analysis, Quantitative PCR, Ribonuclease protection assays. The following cell types are provided for illustrative purposes, but other cell types can be routinely used, provided that the target is expressed in the cell type chosen.
   Cells were cultured in the appropriate medium as described below and maintained at 37° C. at 95-98% humidity and 5% CO₂. Cells were routinely passaged 2-3 times weekly.
   Hepa1-6: Murine liver cell line Hepa1-6 was purchased from ATCC and cultured in DMEM (Sigma) with 10% FBS+Glutamax I+Gentamicin.
   PC3: Human prostate cancer cell line PC3 was purchased from ATCC and cultured in Eagle MEM (Sigma) with 10% FBS+Glutamax I+Gentamicin.
   RAW264.7: Murine monocyte/macrophage cell line RAW264.7 was purchased from ATCC and cultured in Eagle MEM (Sigma) with 10% FBS+Glutamax I+Gentamicin.

### List of Oligonucleotides:

| | | | SEQ |
|---|---|---|---|
| Target | Sequence | Design | ID No |
| FABP4 | GCAtcacacatttTGT | 16-mer, 3-10-3 design | 118 |
| FABP4 | GCAtcacacatTTT | 14-mer, 3-8-3 design | 122 |
| FABP4 | TTCactggagacAAG | 15-mer, 3-9-3 design | 119 |
| FABP4 | TTTcactggagaCAA | 15-mer, 3-9-3 design | 120 |
| FABP4 | GTTttcactggagACA | 16 mer, 3-10-3 design | 123 |
| FABP4 | TCGttttctcttTAT | 15-mer, 3-9-3 design | 117 |
| FABP4 | TCTcgttttctctTTA | 16 mer, 3-10-3 design | 121 |

### Example 1

### In Vitro Model: Treatment with Antisense Oligonucleotide

Cell culturing and transfections: Hepa1-6 cells, PC3 or RAW264.7 were seeded in 6-well plates at 37° C. (5% CO₂) in growth media supplemented with 10% FBS, Glutamax I and Gentamicin. When the cells were 60-70% confluent, they were transfected in duplicates with different concentrations of oligonucleotides (0.04-25 nM) using Lipofectamine 2000 (5 μg/ml). Transfections were carried out essentially as described by Dean et al. (1994, JBC 269:16416-16424). In short, cells were incubated for 10 min. with Lipofectamine in OptiMEM followed by addition of oligonucleotide to a total volume of 0.5 ml transfection mix per well. After 4 hours, the transfection mix was removed, cells were washed and grown at 37° C. for approximately 20 hours Cells were then harvested for protein and RNA analysis.

### Example 2

### In Vitro Model: Extraction of RNA and cDNA Synthesis

### Total RNA Isolation

Total RNA was isolated using RNeasy mini kit (Qiagen). Cells were washed with PBS, and Cell Lysis Buffer (RTL, Qiagen) supplemented with 1% mercaptoethanol was added directly to the wells. After a few minutes, the samples were processed according to manufacturer's instructions.

### First Strand Synthesis

First strand synthesis was performed using either OmniScript Reverse Transcriptase kit or M-MLV Reverse transcriptase (essentially as described by manufacturer (Ambion)) according to the manufacturer's instructions (Qiagen). When using OmniScript Reverse Transcriptase 0.5 μg total RNA each sample, was adjusted to 12 μl and mixed with 0.2 μl poly (dT)₁₂₋₁₈ (0.5 μg/μl) (Life Technologies), 2 μl dNTP mix (5 mM each), 2 μl 10×RT buffer, 0.5 μl RNAguard™ RNase Inhibitor (33 units/ml, Amersham) and 1 μl OmniScript Reverse Transcriptase followed by incubation at 37° C. for 60 min. and heat inactivation at 93° C. for 5 min.
When first strand synthesis was performed using random decamers and M-MLV-Reverse Transcriptase (essentially as described by manufacturer (Ambion)) 0.25 μg total RNA of each sample was adjusted to 10.8 μl in H₂O. 2 μl decamers and 2 μl dNTP mix (2.5 mM each) was added. Samples were heated to 70° C. for 3 min. and cooled immediately in ice water and added 3.25 μl of a mix containing (2 μl 10×RT buffer; 1 μl M-MLV Reverse Transcriptase; 0.25 μl RNAase inhibitor). cDNA is synthesized at 42° C. for 60 min followed by heating inactivation step at 95° C. for 10 min and finally cooled to 4° C.

### Example 3

### In Vitro and In Vivo Model: Analysis of Oligonucleotide Inhibition of FABP4 Expression by Real-Time PCR

Antisense modulation of FABP4 mRNA expression can be assayed in a variety of ways known in the art. For example, FABP4 mRNA levels can be quantified by, e.g., Northern blot analysis, competitive polymerase chain reaction (PCR), or real-time PCR. Real-time quantitative PCR is presently preferred. RNA analysis can be performed on total cellular RNA or mRNA.
Methods of RNA isolation and RNA analysis such as Northern blot analysis are routine in the art and is taught in, for example, Current Protocols in Molecular Biology, John Wiley and Sons.
Real-time quantitative (PCR) can be conveniently accomplished using the commercially iQ Multi-Color Real Time PCR Detection System available from BioRAD or 7500Fast Real-Time PCR System from Applied Biosystem. Real-time Quantitative PCR is a technique well known in the art and is taught in for example Heid et al. Real time quantitative PCR, Genome Research (1996), 6: 986-994.
Real-Time Quantitative PCR Analysis of FABP4 mRNA Levels
To determine the relative mouse FABP4 mRNA level in treated and untreated samples, the generated cDNA was used in quantitative PCR analysis using a 7500Fast Real-Time PCR System from Applied Biosystems. The FABP4 mRNA expression was quantified generally as described by the manufacturer. In brief, 4 μl of cDNA was added 6 μl of a mastermix containing Taqman Fast Universal PCR master mix and a primer-probe mix available from Applied Biosystems.
All samples were run in duplets and correlated to a 2-fold dilution series generated from cDNA made from the respective cell line. Relative quantities of FABP4 mRNA were determined from the calculated threshold cycle using the Sequence Detection Software from Applied Biosystems and normalised to the relative quantities on GAPDH mRNA.

### Example 4

### In Vitro Analysis: Dose Response in Cell Culture (Murine Hepatocyte Cell Line Hepa 1-6, Human Prostate Cancer Cell Line Pc3 and Murine Monocyte/Macrophage Cells Line RAW264.7)/Antisense Inhibition of FABP4 Expression

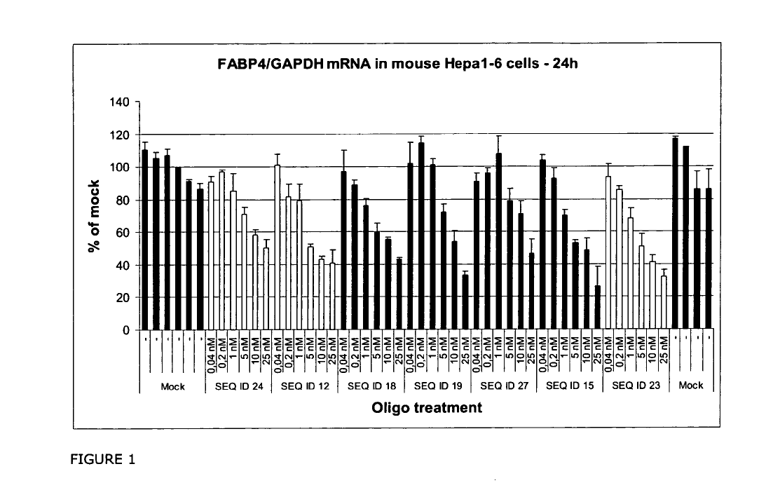

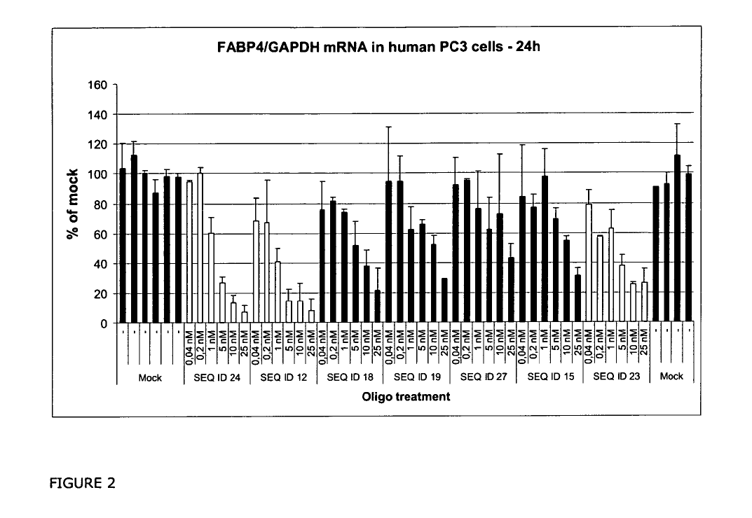

In accordance with the present invention, a series of oligonucleotides were designed to target different regions of the human and murine FABP4 mRNA. See Table 1: Oligonucleotide compounds (marked in bold) were evaluated for their potential to knockdown FABP4 mRNA in human prostate cancer cells (PC3), murine hepatocytes (Hepa 1-6) and murine monocyte/macrophage cells (RAW264.7) following lipid-assisted uptake of SEQ ID NO: 12 (3896), 15 (3900), 18 (3897), 19 (3898), 23 (3901), 24 (3895) and 27(3899) (FIG. 1, FIG. 2, FIG. 3). The experiment was performed as described in examples 1-3. The results showed very potent down regulation (30-50%, 95-60% and >60% in Hep 1-6, PC3 and RAW264.7, respectively) with 25 nM for all compounds. However, at 1 nM only 1 compound resulted in a FABP4 mRNA down regulation as high as 50% in PC3 cells (SEQ ID NO: 12), which is a very potent down regulation (FIG. 2).
The expression of FABP-4 was examined after lipofectamine transfection with 0.04, 0.2, 1, 5, 10 and 25 nM oligonucleotide solution. RNA was isolated from the cells and the expression of FABP-4 mRNA was determined by qPCR as described in examples 1-3.
In Hepa 1-6 (mouse hepatoma cell line) SEQ ID NO. 12, SEQ ID NO. 15 and SEQ ID NO. 23 were the most potent oligonucleotides with IC50 at 5, 5 and between 1 and 5 nM, respectively. Due to lower transfection efficiency in the Hepa 1-6 cells compared to PC3 cells the IC50's is generally higher (FIG. 1).
In PC3 (human prostate cancer cell line) SEQ ID NO. 24, SEQ ID NO. 12, and SEQ ID NO. 23, were shown to be the most potent oligonucleotides to down regulate FABP-4 mRNA with IC50 of 1, 0.2 and 1 nM, respectively (FIG. 2)
Screening of our oligonucleotides in RAW264.7 was made to validate the effect on FABP4 mRNA expression (FIG. 3). RAW264.7 is a murine monocyte/macrophage cell line expressing FABP4. The oligonucleotides SEQ ID NO. 18, 19 and 15 had an IC50 around 5 nM in these cells, whereas SEQ ID NO. 12, and 23 had an IC50 of 5-10 nM, for SEQ ID NO. 27, the IC50 was between 10 and 25 nM and for SEQ ID NO. 24, the IC50 was >25 nM.

### Example 5

### Oligonucleotide Sequences for Down-Regulation of FABP4

| | | | | 100% | 100% | |
|---|---|---|---|---|---|---|
| mRNA | mRNA | | | human | mouse | SEQ |
| target | target | Oligo | | target | target | ID |
| Start | End | length | Oligo sequene | mRNAs | mRNAs | No |
| 41 | 54 | 14 | GCATCACACATTTT | 1 | 1 | 12 |
| 116 | 130 | 15 | GGCAAAGCCCACTCC | 1 | 1 | 13 |
| 118 | 132 | 15 | GTGGCAAAGCCCACT | 1 | 1 | 14 |
| 356 | 370 | 15 | TCGTTTTCTCTTTAT | 1 | 1 | 15 |
| 357 | 371 | 15 | CTCGTTTTCTCTTTA | 1 | 1 | 16 |
| 40 | 54 | 15 | GCATCACACATTTTG | 1 | 1 | 17 |
| 74 | 88 | 15 | TTCACTGGAGACAAG | 1 | 1 | 18 |
| 75 | 89 | 15 | TTTCACTGGAGACAA | 1 | 1 | 19 |
| 116 | 131 | 16 | TGGCAAAGCCCACTC | 1 | 1 | 20 |
| | | | C | | | |
| 117 | 132 | 16 | GTGGCAAAGCCCACT | 1 | 1 | 21 |
| | | | C | | | |
| 356 | 371 | 16 | CTCGTTTTCTCTTTA | 1 | 1 | 22 |
| | | | T | | | |
| 357 | 372 | 16 | TCTCGTTTTCTCTTT | 1 | 1 | 23 |
| | | | A | | | |
| 39 | 54 | 16 | GCATCACACATTTTG | 1 | 1 | 24 |
| | | | T | | | |
| 74 | 89 | 16 | TTTCACTGGAGACAA | 1 | 1 | 25 |
| | | | G | | | |
| 75 | 90 | 16 | TTTTCACTGGAGACA | 1 | 1 | 26 |
| | | | A | | | |
| 76 | 91 | 16 | GTTTTCACTGGAGAC | 1 | 1 | 27 |
| | | | A | | | |
| 161 | 174 | 14 | CTGATGATCATGTT | 1 | 3 | 28 |
| 356 | 369 | 14 | CGTTTTCTCTTTAT | 1 | 3 | 29 |
| 358 | 371 | 14 | CTCGTTTTCTCTTT | 1 | 3 | 30 |
| 358 | 372 | 15 | TCTCGTTTTCTCTTT | 1 | 3 | 31 |
| 227 | 239 | 13 | TGAAGGAAATCTC | 1 | 4 | 32 |
| 359 | 372 | 14 | TCTCGTTTTCTCTT | 1 | 4 | 33 |
| 357 | 370 | 14 | TCGTTTTCTCTTTA | 2 | 1 | 34 |
| 40 | 53 | 14 | CATCACACATTTTG | 2 | 1 | 35 |
| 76 | 89 | 14 | TTTCACTGGAGACA | 2 | 1 | 36 |
| 80 | 93 | 14 | AAGTTTTCACTGGA | 2 | 1 | 37 |
| 39 | 53 | 15 | CATCACACATTTTGT | 2 | 1 | 38 |
| 76 | 90 | 15 | TTTTCACTGGAGACA | 2 | 1 | 39 |
| 77 | 91 | 15 | GTTTTCACTGGAGAC | 2 | 1 | 40 |
| 78 | 92 | 15 | AGTTTTCACTGGAGA | 2 | 1 | 41 |
| 79 | 93 | 15 | AAGTTTTCACTGGAG | 2 | 1 | 42 |
| 77 | 92 | 16 | AGTTTTCACTGGAGA | 2 | 1 | 43 |
| | | | C | | | |
| 78 | 93 | 16 | AAGTTTTCACTGGAG | 2 | 1 | 44 |
| | | | A | | | |
| 118 | 131 | 14 | TGGCAAAGCCCACT | 2 | 2 | 45 |
| 39 | 52 | 14 | ATCACACATTTTGT | 2 | 2 | 46 |
| 79 | 92 | 14 | AGTTTTCACTGGAG | 2 | 2 | 47 |
| 117 | 131 | 15 | TGGCAAAGCCCACTC | 2 | 2 | 48 |
| 42 | 54 | 13 | GCATCACACATTT | 2 | 3 | 49 |
| 117 | 130 | 14 | GGCAAAGCCCACTC | 2 | 3 | 50 |
| 74 | 87 | 14 | TCACTGGAGACAAG | 2 | 3 | 51 |
| 75 | 88 | 14 | TTCACTGGAGACAA | 2 | 3 | 52 |
| 357 | 369 | 13 | CGTTTTCTCTTTA | 2 | 5 | 53 |
| 383 | 395 | 13 | ATTCCACCACCAG | 2 | 6 | 54 |
| 383 | 396 | 14 | CATTCCACCACCAG | 2 | 6 | 55 |
| 162 | 174 | 13 | CTGATGATCATGT | 3 | 4 | 56 |
| 359 | 371 | 13 | CTCGTTTTCTCTT | 3 | 6 | 57 |
| 360 | 372 | 13 | TCTCGTTTTCTCT | 3 | 6 | 58 |
| 77 | 90 | 14 | TTTTCACTGGAGAC | 4 | 1 | 59 |
| 40 | 52 | 13 | ATCACACATTTTG | 4 | 2 | 60 |
| 78 | 91 | 14 | GTTTTCACTGGAGA | 4 | 2 | 61 |
| 39 | 51 | 13 | TCACACATTTTGT | 4 | 3 | 62 |
| 80 | 92 | 13 | AGTTTTCACTGGA | 4 | 3 | 63 |
| 358 | 370 | 13 | TCGTTTTCTCTTT | 4 | 4 | 64 |
| 79 | 91 | 13 | GTTTTCACTGGAG | 4 | 6 | 65 |
| 119 | 132 | 14 | GTGGCAAAGCCCAC | 4 | 6 | 66 |
| 75 | 87 | 13 | TCACTGGAGACAA | 4 | 7 | 67 |
| 161 | 173 | 13 | TGATGATCATGTT | 4 | 8 | 68 |
| 81 | 93 | 13 | AAGTTTTCACTGG | 5 | 1 | 69 |
| 118 | 130 | 13 | GGCAAAGCCCACT | 5 | 3 | 70 |
| 43 | 54 | 12 | GCATCACACATT | 5 | 6 | 71 |
| 227 | 238 | 12 | GAAGGAAATCTC | 5 | 8 | 72 |
| 119 | 131 | 13 | TGGCAAAGCCCAC | 5 | 8 | 73 |
| 356 | 368 | 13 | GTTTTCTCTTTAT | 5 | 9 | 74 |
| 41 | 53 | 13 | CATCACACATTTT | 6 | 3 | 75 |
| 361 | 372 | 12 | TCTCGTTTTCTC | 6 | 8 | 76 |
| 384 | 396 | 13 | CATTCCACCACCA | 6 | 10 | 77 |
| 77 | 89 | 13 | TTTCACTGGAGAC | 7 | 2 | 78 |
| 74 | 86 | 13 | CACTGGAGACAAG | 7 | 3 | 79 |
| 360 | 371 | 12 | CTCGTTTTCTCT | 7 | 10 | 80 |
| 76 | 88 | 13 | TTCACTGGAGACA | 8 | 6 | 81 |
| 120 | 132 | 13 | GTGGCAAAGCCCA | 8 | 7 | 82 |
| 384 | 395 | 12 | ATTCCACCACCA | 8 | 14 | 83 |
| 161 | 172 | 12 | GATGATCATGTT | 9 | 11 | 84 |
| 78 | 90 | 13 | TTTTCACTGGAGA | 10 | 2 | 85 |
| 163 | 174 | 12 | CTGATGATCATG | 10 | 9 | 86 |
| 358 | 369 | 12 | CGTTTTCTCTTT | 10 | 11 | 87 |
| 359 | 370 | 12 | TCGTTTTCTCTT | 10 | 12 | 88 |
| 162 | 173 | 12 | TGATGATCATGT | 10 | 17 | 89 |
| 228 | 239 | 12 | TGAAGGAAATCT | 11 | 14 | 90 |
| 74 | 85 | 12 | ACTGGAGACAAG | 12 | 6 | 91 |
| 42 | 53 | 12 | CATCACACATTT | 12 | 10 | 92 |
| 75 | 86 | 12 | CACTGGAGACAA | 12 | 13 | 93 |
| 82 | 93 | 12 | AAGTTTTCACTG | 12 | 13 | 94 |
| 40 | 51 | 12 | TCACACATTTTG | 13 | 4 | 95 |
| 39 | 50 | 12 | CACACATTTTGT | 13 | 8 | 96 |
| 119 | 130 | 12 | GGCAAAGCCCAC | 13 | 12 | 97 |
| 80 | 91 | 12 | GTTTTCACTGGA | 13 | 15 | 98 |
| 121 | 132 | 12 | GTGGCAAAGCCC | 14 | 12 | 99 |
| 120 | 131 | 12 | TGGCAAAGCCCA | 14 | 21 | 100 |
| 81 | 92 | 12 | AGTTTTCACTGG | 15 | 6 | 101 |
| 79 | 90 | 12 | TTTTCACTGGAG | 15 | 11 | 102 |
| 385 | 396 | 12 | CATTCCACCACC | 15 | 12 | 103 |
| 77 | 88 | 12 | TTCACTGGAGAC | 16 | 15 | 104 |
| 41 | 52 | 12 | ATCACACATTTT | 17 | 8 | 105 |
| 357 | 368 | 12 | GTTTTCTCTTTA | 18 | 21 | 106 |
| 76 | 87 | 12 | TCACTGGAGACA | 18 | 23 | 107 |
| 116 | 129 | 14 | GCAAAGCCCACTCC | 21 | 3 | 108 |
| 78 | 89 | 12 | TTTCACTGGAGA | 24 | 10 | 109 |
| 116 | 128 | 13 | CAAAGCCCACTCC | 26 | 6 | 110 |
| 383 | 394 | 12 | TTCCACCACCAG | 27 | 18 | 111 |
| 117 | 129 | 13 | GCAAAGCCCACTC | 28 | 47 | 112 |
| 116 | 127 | 12 | AAAGCCCACTCC | 30 | 11 | 113 |
| 118 | 129 | 12 | GCAAAGCCCACT | 35 | 50 | 114 |
| 356 | 367 | 12 | TTTTCTCTTTAT | 42 | 56 | 115 |
| 117 | 128 | 12 | CAAAGCCCACTC | 43 | 55 | 116 |

1. An oligomer, of between 10-50 nucleobases in length which comprises a contiguous nucleobase sequence of a total of between 10-50 nucleobases, wherein said contiguous nucleobase sequence is at least 80% homologous to a corresponding region of a nucleic acid which encodes a mammalian FABP4; wherein said oligomer is for use as a medicament or for use in a pharmaceutical composition.

2. The oligomer according to claim 1, wherein the contiguous nucleobase sequence comprises no more than 3 mismatches to the corresponding region of a nucleic acid which encodes a mammalian FABP4.

3. The oligomer according to claim 2, wherein said contiguous nucleobase sequence comprises no more than a single mismatch to the corresponding region of a nucleic acid which encodes a mammalian FABP4.

4. The oligomer according to claim 3, wherein said contiguous nucleobase sequence comprises no mismatches to the corresponding region of a nucleic acid which encodes a mammalian FABP4.

5. The oligomer according to claim 1, wherein the nucleobase sequence of the oligomer consists of the contiguous nucleobase sequence.

6. The oligomer according to claim 1, wherein the nucleic acid which encodes a mammalian FABP4 is human FABP4, or a naturally occurring allelic variant thereof.

7. The oligomer according to claim 1, wherein the nucleic acid which encodes a mammalian FABP4 selected from the group consisting of a nucleic acid which encodes a rodent FABP4 and a non-human primate FABP4.

8. The oligomer according to claim 1, wherein the contiguous nucleobase sequence is complementary to a corresponding region of both the human FABP4 nucleic acid sequence and a non-human mammalian FABP4 nucleic acid sequence.

9. The oligomer according to claim 1, wherein the contiguous nucleobase sequence is complementary to a corresponding region of both the human FABP4 nucleic acid sequence, SEQ ID NO 1 and the mouse FABP4 nucleic acid sequence, SEQ ID NO 3.

10. The oligomer according to claim 1, wherein the contiguous nucleobase sequence comprises a contiguous sub-sequence of at least 7 nucleobase residues which, when formed in a duplex with the complementary FABP4 target RNA is capable of recruiting RNaseH.

11. The oligomer according to claim 10, wherein the contiguous nucleobase sequence comprises of a contiguous sub-sequence of at least 8, at least 9 or at least 10 nucleobase residues which, when formed in a duplex with the complementary FABP4 target RNA is capable of recruiting RNaseH.

12. The oligomer according to claim 11 wherein said contiguous sub-sequence is at least 12 nucleobases or at least 14 nucleobases in length which, when formed in a duplex with the complementary FABP4 target RNA is capable of recruiting RNaseH.

13. The oligomer according to claim 1 wherein said oligomer is conjugated with one or more non-nucleobase compounds.

14. The oligomer according to claim 1, wherein said oligomer has a length of between 10-22 nucleobases.

15. The oligomer according to claim 1, wherein said oligomer has a length of between 12-18 nucleobases.

16. The oligomer according to claim 1, wherein said oligomer has a length of 14, 15 or 16 nucleobases.

17. The oligomer according to claim 1, wherein said continuous nucleobase sequence corresponds to a contiguous nucleotide sequence present in a nucleic acid sequence selected from the group consisting of SEQ ID NOs: 5, 6, 7, 8, 9, 10, 11, 12 and 23.

18. The oligomer according to claim 1, wherein the oligomer or contiguous nucleobase sequence is selected from the group consisting of SEQ ID NO 118, 122, 119, 120, 123, 117 and 121.

19. The oligomer according to claim 1 wherein said oligomer is single stranded.

20. The oligomer according to claim 1, wherein said contiguous nucleobase sequence comprises at least one affinity enhancing nucleotide analogue.

21. The oligomer according to claim 20, wherein said contiguous nucleobase sequence comprises a total of 2, 3, 4, 5, 6, 7, 8, 9 or 10 affinity enhancing nucleotide analogues.

22. The oligomer according to claim 1 which comprises at least one affinity enhancing nucleotide analogue and wherein the remaining nucleobases are selected from the group consisting of DNA nucleotides and RNA nucleotides.

23. The oligomer according to claim 1, wherein the oligomer comprises of a sequence of nucleobases of formula, in 5′ to 3′ direction, A-B-C, wherein:
A comprises of at least one nucleotide analogue;
B comprises at least five consecutive nucleobases which are capable of recruiting RNaseH and;
C comprises at least one nucleotide analogue.

24. The oligomer according to claim 23, wherein region A consists or comprises of 2, 3 or 4 consecutive nucleotide analogues.

25. The oligomer according to claim 23, wherein region B comprises of 7, 8, 9 or 10 consecutive DNA nucleotides or equivalent nucleobases which are capable of recruiting RNaseH when formed in a duplex with a complementary RNA.

26. The oligomer according to claim 1, wherein region C comprises of 2, 3 or 4 consecutive nucleotide analogues.

27. (canceled)

28. The oligomer according to claim 23, wherein:
A comprises of 3 consecutive nucleotide analogues;
B comprises of 7, 8, 9 or 10 consecutive DNA nucleotides or equivalent nucleobases which are capable of recruiting RNaseH when formed in a duplex with a complementary RNA, such as the FAPB4 mRNA target;
C comprises 3 consecutive nucleotide analogues.

29. The oligomer according to claim 23, wherein B comprises at least one LNA nucleobase which is in the alpha-L configuration.

30. The oligomer according to claim 1, wherein the nucleotide analogue(s) are selected from the group consisting of: Locked Nucleic Acid (LNA) units; 2′-O-alkyl-RNA units, 2′-OMe-RNA units, 2′-amino-DNA units, 2′-fluoro-DNA units, PNA units, HNA units, and INA units.

31. The oligomer according to claim 30 wherein all the nucleotide analogue(s) are LNA units.

32. The oligomer according to claim 1, which comprises 1, 2, 3, 4, 5, 6, 7, 8, 9, or 10 LNA.

33. The oligomer according to claim 30, wherein the LNAs are selected from oxy-LNA, thio-LNA, and amino-LNA, in either of the beta-D and alpha-L configurations or combinations thereof.

34. The oligomer according to claim 33, wherein the LNAs are all β-D-oxy-LNA.

35. The oligomer according to claim 23, wherein the nucleotide analogues or nucleobases of regions A and C are β-D-oxy-LNA.

36. The oligomer according to claim 1, wherein at least one of the nucleobases present in the oligomers a modified nucleobase is selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

37. The oligomer according to claim 1, wherein said oligomer hybridizes with a corresponding mammalian FABP4 mRNA with a Tm of at least 50° C.

38. The oligomer according to claim 1, wherein said oligomer hybridizes with a corresponding mammalian FABP4 mRNA with a Tm of no greater than 80° C.

39. The oligomer according to claim 1, wherein the internucleoside linkages are selected from the group consisting of: phosphodiester, phosphorothioate and boranophosphate.

40. The oligomer according to claim 39, wherein the oligomer comprises at least one phosphorothioate internucleoside linkage.

41. The oligomer according to claim 41, wherein the internucleoside linkages adjacent to or between DNA or RNA units, or within region B are phosphorothioate linkages.

42. The oligomer according to claim 40, wherein the linkages between at least one pair of consecutive nucleotide analogues is a phosphodiester linkage.

43. The oligomer according to claim 40, wherein all the linkages between consecutive nucleotide analogues are phosphodiester linkages.

44. The oligomer according to claim 40 wherein all the internucleoside linkages are phosphorothioate linkages.

45. A conjugate comprising the oligomer according to claim 1 and at least one non-nucleotide or non-polynucleotide moiety covalently attached to said oligomer.

46. A pharmaceutical composition comprising an oligomer as defined in claim 1 and a pharmaceutically acceptable diluent, carrier, salt or adjuvant.

47. A pharmaceutical composition according to claim 46, wherein the oligomer is constituted as a pro-drug.

48. A pharmaceutical composition according to claim 46, which further comprises a further therapeutic agent selected from the group consisting of: an Apo-B-100 antagomir, a PCSK9 antagomir, a statin, a fibrate, a thioazolidinedione, an anti-inflamatory compound and an antiviral compound.

49. A method of treating an inflammatory or metabolic disease comprising administering to a patient in need thereof the oligomer of claim 1.

50. A method for treating an inflammatory disorder comprising administering the oligomer of claim 1 to a patient in need thereof.

51. A method of reducing the expression of FABP4 in a cell or a tissue, comprising the step of contacting said cell or tissue with an effective amount of the oligomer of claim 1 so that expression of FABP4 is reduced.

52. A method of (i) reducing the level of blood serum cholesterol or ii) reducing the level of blood serum LDL-cholesterol, or iii) for improving the HDL/LDL ratio, in a patient, the method comprising the step of administering the oligomer of claim 1 to the patient.

53. A method of lowering the plasma triglyceride in a patient, the method comprising the step of administering the oligomer of claim 1 to the patient so that the blood serum triglyceride level is reduced.

54. A method of treating obesity in a patient, the method comprising the step of administering the oligomer of claim 1 to the patient in need of treatment so that the body weight of the patient is reduced.

55. A method of treating insulin resistance in a patient, the method comprising the step of administering the oligomer of claim 1 to the patient in need of treatment so that the patients sensitivity to insulin is increased.

56. A method of treating type II diabetes in a patient, the method comprising the step of administering the oligomer of claim 1 to the patient.

57. A method for treating a metabolic disorder, said method comprising administering the oligomer of claim 1 to a patient in need thereof.

58. The oligomer of claim 23, further comprising:
D which comprises one or more DNA nucleotides.

59. The oligomer of claim 58, wherein:
A comprises of 3 consecutive nucleotide analogues;
B comprises of 7, 8, 9 or 10 consecutive DNA nucleotides or equivalent nucleobases which are capable of recruiting RNaseH when formed in a duplex with a complementary RNA;
C comprises 3 consecutive nucleotide analogues; and
D comprises one or two DNA nucleotides.

60. A pharmaceutical composition comprising the conjugate of claim 45, and a pharmaceutically acceptable diluent, carrier, salt or adjuvant.

61. A method of treating an inflammatory or metabolic disease comprising administering to a patient in need thereof the conjugate of claim 45.

62. A method for treating an inflammatory disorder comprising administering the conjugate of claim 45 to a patient in need thereof.

63. A method of reducing the expression of FABP4 in a cell or a tissue, comprising the step of contacting said cell or tissue with an effective amount of the conjugate of claim 45 so that expression of FABP4 is reduced.

64. A method of (i) reducing the level of blood serum cholesterol or ii) reducing the level of blood serum LDL-cholesterol, or iii) for improving the HDL/LDL ratio, in a patient, the method comprising the step of administering the conjugate of claim 45 to the patient.

65. A method of lowering the plasma triglyceride in a patient, the method comprising the step of administering the conjugate of claim 45 to the patient so that the blood serum triglyceride level is reduced.

66. A method of treating obesity in a patient, the method comprising the step of administering the conjugate of claim 45 to the patient in need of treatment so that the body weight of the patient is reduced.

67. A method of treating insulin resistance in a patient, the method comprising the step of administering the conjugate of claim 45 to the patient in need of treatment so that the patients sensitivity to insulin is increased.

68. A method of treating type II diabetes in a patient, the method comprising the step of administering the conjugate of claim 45 to the patient.

69. A method for treating a metabolic disorder comprising administering an effective amount the conjugate of claim 45 to a patient in need thereof.
